(19) United States
(12) Patent Application Publication
(10) Pub. No.: US 2022/0388002 A1
(43) Pub. Date: Dec. 8, 2022

(54) MICROFLUIDIC DEVICE AND USES THEREOF

(71) Applicant: CASE WESTERN RESERVE UNIVERSITY, Cleveland, OH (US)

(72) Inventors: Umut GURKAN, Cleveland, OH (US); Erdem KUCUKAL, Cleveland, OH (US); Yuncheng MAN, Cleveland, OH (US)

(21) Appl. No.: 17/776,112

(22) PCT Filed: Nov. 12, 2020
(86) PCT No.: PCT/US2020/060227

§ 371 Date: May 11, 2022

Related U.S. Application Data
(60) Provisional application No. 62/933,781, filed on Nov. 11, 2019.
(60) Provisional application No. 63/019,710, filed on May 4, 2020.

(51) Int. Cl. B01L 3/00; A61B 5/15

(52) U.S. Cl. CPC B01L 3/502753; A61B 5/150022; A61B 5/150358; B01L 2200/0668; B01L 2300/0816; B01L 2300/0877; B01L 2400/086

(57) ABSTRACT

A microfluidic device includes at least one microchannel with a plurality of micropillar arrays provided along a length of the microchannel. Each micropillar array defines a plurality microcapillaries having cross sectional area, and the cross sectional area of the microcapillaries defined by each micropillar array decreases in a direction of fluid flow through the microchannel.

### RELATED APPLICATION

This application claims priority from U.S. Provisional Application No. 62/933,781, filed Nov. 11, 2019 and 63/019,710 filed May 4, 2020, the subject matter of which are incorporated herein by reference in their entirety.

### GOVERNMENT FUNDING

This invention was made with government support under Grant No. CMMI-1552782 awarded by The National Science Foundation. The United States government has certain rights to the invention.

### BACKGROUND

Deformability of red blood cells (RBCs) is critical for continuous blood flow in the microcirculation. Healthy RBCs are highly deformable, allowing them to pass through minuscule blood vessels to facilitate oxygen delivery. However, in RBC disorders, such as sickle cell disease (SCD) and malaria, deformability is pathologically altered. In SCD, abnormal RBC deformability is induced by intracellular polymerization of sickle hemoglobin (HbS) under deoxygenated conditions. Impaired RBC deformability significantly contributes to the pathophysiological hallmarks of this disorder, including hemolysis, inflammation, microvascular occlusion and consequent organ failures. In malaria, aberrant RBC deformability is caused by membrane-protein network modifications associated with Plasmodium parasites. Conventional techniques developed for RBC deformability assessment include atomic force microscopy, micropipette aspiration, optical tweezers, and osmotic gradient ektacytometry. Even though these techniques have proven useful in assessing RBC deformability in vitro, they are time and skill intensive, low-throughput, can exhibit limited physiological relevance and clinical utility.

Microfluidic technologies have been developed to better recapitulate in vivo microvascular environment and to probe RBC deformability in precisely controlled flow conditions at both bulk and single-cell level. Single-cell microfluidic approaches have been used to measure RBC deformability by assessing micro-constriction obstruction, shear deformation, membrane fluctuation, lateral margination, cell velocity, transient time, electrical impedance, and electrical deformation. Single-cell approaches are low-throughput and yield limited information on a small fraction of cells, which may not represent the overall cell population. Bulk-cell microfluidic approaches have been utilized to characterize the deformability of bulk RBCs by measuring the average cell retention, cell elongation, and average cell transient velocity. However, none of these techniques fully mimic the capillary bed architecture or provide a direct assessment of the pathophysiological impact of impaired RBC deformability on the microcirculation.

### SUMMARY

This disclosure describes a microfluidic device and system for measuring cell deformability, occlusion, and/or adhesion both at the single cell level and in bulk, and particularly relates to a microfluidic device and system for assessing red blood cell (RBC) deformability and microvascular occlusion rate associated with RBC deformability, occlusion, and adhesion. In some embodiments, the microfluidic device can mimic architectural features associated with capillary beds of vasculature or microvasculature of a subject. The microfluidic device can include a plurality of micropillar arrays within a microchannel that define a plurality of microcapillaries that mimic capillary networks of microvasculature of a subject. These microcapillaries can be engineered to retain RBCs with impaired deformability, such that more abnormal RBCs will occlude wide upstream microcapillaries, while those with moderate impairment will occlude finer downstream microcapillaries within the microchannel. The microchannel can also be designed with two wide side or outer openings or passages to mimic arteriovenous anastomoses, which act as shunts in the capillary bed in vivo. These side anastomoses can prevent complete blockade of flow in the microchannel, and enable testing of clinical blood samples with near-physiological hematocrit levels.

Advantageously, RBCs perfused through the microfluidic device experience a wide spectrum of deformations when crossing microcapillaries with different sizes, which recapitulates a more physiologically relevant microenvironment. Additionally, the microfluidic device can examine large numbers of heterogeneous RBCs since the embedded micropillar arrays recapitulate large numbers of microcapillaries with various dimensions, enabling the simultaneous deformability analysis of bulk RBCs at a single-cell level. The visual quantification of occlusions induced by poorly deformable RBCs, which are retained by the microcapillaries within each micropillar array, and the resulting analysis, make the assessment of overall RBC deformability and associated microvascular occlusion possible. Moreover, the microfluidic device can be used to assess very small changes in RBC deformability, under both normoxic (ambient air) and hypoxic conditions, to assess pathologically impaired RBCs in blood. The assessment of pathologically impaired RBCs can be used to assess microvascular health and function of a subject and determine the subject's increased risk of vaso-occlusive crises (VOC) in a range of microcirculatory diseases.

In some embodiments, the microfluidic device can include at least one microchannel that extends through a portion of a housing. The microchannel can be configured to receive a fluid sample that flows along a length of the microchannel from a first end to a second end of the microchannel. The microchannel can include a plurality of micropillar arrays provided along the length of the microchannel. Each micropillar array can define a plurality microcapillaries having a width, height, and cross sectional area. The width and/or cross sectional area of the microcapillaries defined by each micropillar array decreases in a direction of fluid flow through the microchannel.

In some embodiments, the microchannel can include a substantially planar upper surface and a substantially planar lower surface. Micropillars of the plurality of micropillar arrays can extend from upper surface to the lower surface.

In other embodiments, each of the micropillars of the plurality of micropillar arrays can have a substantially rectangular cross section and/or each of the microcapillaries defined by the micropillar arrays can have a substantially rectangular cross section.

In some embodiments, the microchannel includes at least 3, 4, 5, 6, 7, 8, 9, 10 or more micropillar arrays arranged in series and the width, height, and cross sectional areas of the microcapillaries defined by each respective micropillar array can be substantially uniform.

In other embodiments, each micropillar array can include at least 3, 4, 5, 6, 7, 8, 9, 10 or more rows of micropillars. The rows can extend perpendicular to fluid flow and have a substantially similar shape. The distance between each micropillar in a respective row of a respective micropillar array can be substantially the same.

In some embodiments, successive micropillar arrays can be separated from each other in the microchannel by a gap region, which is free of micropillars. The gap region can be of a length that allows cells, such as RBCs, in the fluid sample to recover, at least partially, their shape after passing through the microcapillaries of a respective micropillar array.

In other embodiments, the microchannel can include a micropillar array at the first end that defines a plurality of microcapillaries that can each have a width of about 18 μm to about 22 μm and a cross sectional area of about 200 μm² to about 250 μm². Each successive micropillar array in the direction of fluid flow through the microchannel can define a plurality of microcapillaries that can each have a width and/or a cross sectional area about 5% to about 50% less than a plurality of microcapillaries defined by a preceding micropillar array.

In other embodiments, the microchannel includes a micropillar array at the second end that defines a plurality of microcapillaries that can each have a width of about 3 μm to about 5 μm and a cross sectional area of about 40 μm² to about 50 μm². Each preceding micropillar array opposite the direction of fluid flow through the microchannel can define a plurality of microcapillaries that can each have a width and/or a cross sectional area about 5% to about 50% greater than a plurality of microcapillaries defined by a preceding micropillar array.

In some embodiments, the microchannel can include at least eight micropillar arrays that are provided or configured in series along the length of the microchannel. A micropillar array at the first end can define a plurality of microcapillaries that each have a width of about 18 μm to about 22 μm and a cross sectional area of about 200 μm² to about 250 μm², and a micropillar array at the second end can define a plurality of microcapillaries that each have a width of about 3 μm to about 5 μm and a cross sectional area of about 40 μm² to about 50 μm².

In some embodiments, the width and/or cross sectional area of the plurality of microcapillaries defined by at least one of the plurality of micropillar arrays permits passage of healthy cells in a fluid sample perfused through the microchannel but occludes cells with impaired deformability. The cell can be blood cells, such as red blood cells.

In other embodiments, the width and/or cross sectional area of the plurality of microcapillaries at the second of the microchannel can occlude cells in a fluid sample perfused through the microchannel.

In some embodiments, each of the micropillar arrays can be arranged in an inner portion of the microchannel that extends the length of the microchannel. The microchannel can include two parallel outer or side passages on opposite sides of the inner portion that extend the length of the microchannel. The outer passages can be in fluid communication with the plurality of microcapillaries defined by the plurality micropillar arrays. The outer passages can have cross sectional areas that permit cells in a fluid sample to flow through the microchannel without being occluded and/or obstructed.

In some embodiments, the microchannel can include a substantially planar transparent wall that defines the upper surface or lower surface of the microchannel. The substantially planar transparent wall can permit observation into the microfluidic channel by microscopy.

In other embodiments, the microfluidic device can include a micro-gas exchanger for controlling the oxygen content of a fluid prior to and/or during perfusion of the fluid through the at least one microchannel. The micro-gas exchanger can provide a fluid sample under normoxic or hypoxic conditions to and/or through the at least one microchannel.

In further embodiments, at least one capturing agent can be immobilized on or to a surface of the at least one microchannel. The capturing agent can adhere a cell of interest to the at least one surface of the at least one microchannel when a fluid sample containing cells is passed through the at least one microchannel. The at least one capturing agent can include, for example, at least one of laminin, fibronectin, E-Selectin, P-Selectin, L-selectin, intracellular adhesion molecule 1 (ICAM-1), or vascular cellular adhesion molecule 1 (VCAM-1). The capturing agent can be covalently immobilized to at least one surface of the microchannel with a cross-linker, such as GMBS.

Other embodiments described herein relate to a microfluidic system that includes the microfluidic device described herein. The microfluidic system can further include a pressure pump and a reservoir that are in fluid communication with the at least one microchannel of the microfluidic device. The reservoir can include a fluid sample that includes blood cells. The pressure pump can be configured to provide pressure to the reservoir such that the fluid sample flows through the at least one microchannel.

In some embodiments, the fluid sample can flow through the at least one microchannel at a physiologically relevant shear stress value. The physiologically relevant shear stress value can be about 0.5 dyne/cm² to about 1 dyne/cm².

The microfluidic system can further include an imaging system for measuring the deformability, adherence, and/or number of cells of interest in the at least one microchannel when the fluid sample is passed therethrough. In some embodiments, the imaging system can be used to measure and/or determine the number of occluded cells in the at least one microchannel.

In some embodiments, the imaging system can include a control unit for determining viscosity of the fluid sample. The viscosity of the fluid sample can be determined by measuring the mean flow velocity of the fluid sample as it passes through the microchannel.

Other embodiments described herein relate to a method of assessing microvascular health and function of a subject in need thereof. The method can include perfusing a fluid sample including RBCs from a blood sample of the subject through the at least one microchannel of a microfluidic device described herein. The number of occluded RBCs in the at least one microchannel can then be measured. A RBC occlusive index (ROI) can be generated from the measured number of occluded red blood cells. The ROI can be indicative of increased risk of vaso-occlusive crises (VOC) and/or microvascular health and function of the subject.

In some embodiments, the ROI can be compared to a control value. The subject can have an increased risk of vaso-occlusive crises (VOC) and/or decreased microvascular health and function when the ROI is greater than the control value.

In some embodiments, the fluid sample can be perfused under at least one normoxic or hypoxic conditions and the number of RBCs can be measured using an imaging system.

Still other embodiments relate to a method of assessing the pathology of RBCs. The method can include perfusing a fluid sample including the RBCs through the at least one microchannel of a microfluidic device described herein. The number of occluded red blood cells can be measured in the microchannel. A RBC occlusive index (ROI) can be generated from the measured number of occluded RBCs. The ROI can be indicative of the number of pathologically impaired RBCs.

In some embodiments, the RBCs are from a subject at risk of a vaso-occlusive crises and/or decreased microvascular health and function. The subject has an increased risk of vaso-occlusive crises (VOC) and/or decreased, diminished, and/or aberrant microvascular health and function when the ROI is greater than the control value. In some embodiments, subject can have malaria or sickle cell disease (SCD). In some embodiments, the red blood cells can be from stored blood and/or blood to be transfused and the ROI can be used to determine the fitness or storage lesions of the stored RBCs and/or RBCs to be transfused.

Still other embodiments relate to a method of measuring efficacy of therapeutic agent in modulating blood cell adhesion and/or deformability. The method can include perfusing a fluid sample including blood cells through the at least one microchannel of a microfluidic device described herein. The number of occluded blood cells in the at least one microchannel can then be measured. The therapeutic agent can be added to at least one of the fluid sample prior to perfusion through the at least one microchannel or the at least microchannel before and/or during perfusion of the fluid sample through the at least one microchannel.

In some embodiments, the efficacy of the therapeutic agent can be determined based on the measured number of occluded blood cells. A decrease in the measured number of occluded blood cells compared to a control is indicative of the therapeutic agent having an increased efficacy in decreasing blood cell adhesion and/or increasing blood cell deformability.

Still other embodiments relate to a method of separating plasma from whole blood while mitigating RBC lysis. The method can include sedimenting RBC from the whole blood without lysing the RBCs. In some embodiments, sedimentation can be accelerated by adding fibrinogen to a whole blood sample. The sedimented red blood cells can be separated from plasma in the whole blood sample. A red blood cell stiffener, such as a diamide, can then be added to the plasma and the plasma can be perfused through at least one microchannel of the microfluidic device to occlude any remaining red blood cells.

In some embodiments, the microfluidic device can include a micropillar array at the second end that defines a plurality of microcapillaries that each have cross sectional area effective to occlude the passage of red blood cells but not plasma.

### BRIEF DESCRIPTION OF THE DRAWINGS

FIG. 1 illustrates a schematic drawing of a microfluidic system in an accordance with an embodiment described herein.

FIG. 2 illustrates a schematic drawing of a microfluidic device in accordance with an embodiment described herein. Arrows indicated direction of flow

FIG. 3 illustrates a schematic of a plurality of micropillars in a microchannel of the microfluidic device of FIG. 2. Arrows indicated direction of flow.

FIG. 4 illustrates a schematic of fluid flow through a row a micropillar array of a microchannel. Arrows indicate direction of flow.

FIG. 5 is a flow diagram that illustrates a process for separation of plasma from whole blood without RBC lysis.

FIGS. 6(A-C) illustrate: (A) A schematic representation of the human capillary bed consisting of microcapillaries and arteriovenous anastomoses. (B) OcclusionChip design features nine micropillar arrays with openings ranging from 20 to 4 μm, with ‘arteriovenous anastamotic’ side openings that are 60 μm wide. Inset: SEM images showing the fabricated micropillar arrays, with dimensions in micrometers. Scale bars represent a length of 100 μm and 10 μm, respectively. (C) The assembled microfluidic device is shown.

FIGS. 7(A-I) illustrate an assessment of microvascular occlusion mediated by chemically treated RBCs in the OcclusionChip. (A) A bright field and EGFP overlay image showing microcapillary occlusions induced by poorly deformable RBCs. (B) (Top to bottom) Overview of retained RBCs stiffened by glutaraldehyde with the concentration of 0.02%, 0.04% and 0.08% (w/v) (control not shown) in the microfluidic device. Scale bars represent a length of 500 μm. Arrows indicate flow direction. Dash zones indicate each micropillar array. (C) Schematics of the unique patterns of the retained RBCs stiffened by graded glutaraldehyde. Profiles of occlusions within the four micropillar arrays generated by RBCs exposed to graded concentrations of (D) glutaraldehyde, (E) diamide, and (F) mercuric ion, and the accordingly computed RBC Occlusion Index generated by RBCs exposed to graded concentrations of (G) glutaraldehyde, (H) diamide, and (I) mercuric ion are shown (N=4 in each group). Error bars represent standard error of the mean.

FIGS. 8(A-C) illustrate an assessment of microvascular occlusion mediated by pathologically abnormal RBCs from subjects with sickle cell disease (SCD) in the OcclusionChip. (A) Representative fluorescent image of retained HbSS RBCs across the microfluidic device. Typical morphologies of retained HbSS RBCs within microcapillaries are shown in subsequent insets. Scale bar represents a length of 500 μm, 50 μm, and 10 μm, respectively. Arrow indicates flow direction. (B) Profiles of number of occlusions induced by normal (HbAA) RBCs and SCD (HbSS) RBCs (5 HbAA and 16 HbSS). (C) RBC occlusion index generated by HbSS RBCs is significantly higher compared to HbAA RBCs (p=0.001, Mann-Whitney).

FIGS. 9(A-I) illustrate hypoxia-mediated HbS-carrying RBC sickling and microvascular occlusion in the OcclusionChip. (A) Macroscale view of the OcclusionChip integrated with a custom designed gas exchange tubing and gas exchange microchannel. (B) Schematic of axisymmetric cross section of the gas exchange tubing showing oxygen diffusion. Arrows indicate the flow direction. (C) Schematic of the fabrication of the gas exchange microchannel. Cross-sectional view shows the oxygen level stabilization within the microchannel. (D) Normalized gray-level intensity measured near the outlet of the microchannel when a solution containing the oxygen-sensitive luminescence probe was flushed at 0 s. (E) Identification of hypoxia-mediated cell sickling of HbS-carrying RBCs. (F) Scanning electron microscopy images show hypoxia-enhanced microchannel occlusion confirming that the deformability of HbS-carrying RBCs is mediated by hypoxia. (G) RBC occlusion index generated by HbS-carrying RBCs increased in response to hypoxia while RBC occlusion index generated by HbAA RBCs remained hypoxia non-responsive (4 HbSS and 3 HbSC). (H) Profiles of occlusions induced by HbS-carrying RBCs within the microchannel in normoxic or hypoxic conditions. (I) A strong positive correlation (PCC=0.7636, p=0.046, N=7) was observed between the RBC Occlusion Index and HbS levels of the subjects under hypoxia.

FIG. 10 illustrates spectrum of (normoxic) RBC Occlusion Index revealed by OcclusionChip. These varying ROIs were generated by abnormal RBCs in various conditions including non-specific protein cross-linking (Glutaraldehyde), cytoskeletal-specific stiffening (Diamide), heavy metal toxin (HgCl₂), storage lesion, and circulatory diseases (renal failure, malaria, SCD). Stars (*) at the top of the bars indicate statistically significant difference (*: p<0.05; **: p<0.01; *: p<0.001).

FIGS. 11(A-F) are schematics showing the fabrication process of OcclusionChip. (A) A photomask with designed microchannel features was used to pattern onto a silicon wafer. (B) The wafer was spin-coated with a negative photoresist layer (SU8-2010), soft-baked, and exposed to UV light under the photomask. (C) After post-exposure baking, developing and hard baking, the master wafer was completed. (D) The wafer was covered with PDMS and then cured. (E-F) The cured PDMS block was peeled-off from the master wafer, and two holes were punched as the inlet and the outlet. After cleaned, the PDMS block was bond to a glass slide to complete the assembly of OcclusionChip.

FIGS. 12(A-B) illustrate OcclusionChip experimental setup. (A) A cost-effective inflation syringe was employed as the pressure source. The pressure was adjusted by applying the indicator to the end of the green zone, which maintained a constant pressure of 60 cm H₂O at the inlet of the microchannel. (B) Pre-prepared RBC suspension was input into the sample reservoir and connected to the inlet of the OcclusionChip. The pressure was controlled by valve 1. Valve 2 was used for back flow prevention. Switching off the two valves can realize the solution change. A waste reservoir was connected to the outlet of the OcclusionChip.

FIG. 13 is a close-up view of a typical deformable RBC with the characteristic biconcave shape traversing the 4-μm microcapillary. The RBC folded as it passed through a 4-μm microcapillary formed by the micropillars, and then underwent shape recovery as soon as it exited the microcapillary.

FIGS. 14(A-B) illustrate velocity and shear environment created by the microcapillary features. (A) Velocity profiles of the initial flow condition in micropillar arrays with 20-μm (left) and 4-μm (right) microcapillaries. (B) Maximum velocity and shear rate contours across the microchannel. Arrow indicates flow direction.

FIGS. 15(A-C) illustrate validation of OcclusionChip micropillar array functionality using rigid fluorescent beads. This initial assay primarily focused on the verification of the retention mechanism of the OcclusionChip. (A) The OcclusionChip was placed on the automated microscope stage for high resolution image recording. A visually clear line formed by 10-μm microbeads. (B) Close-up view of microbeads captured in the micropillar array with 8-μm microcapillaries. Scale bar represents a length of 20 μm. (C) Fluorescent images show that microbeads are able to clear the microcapillaries larger than or equal to their diameters but can block those smaller than their outer diameters. (From top to bottom) Tests were performed with 10-μm, 6-μm, and 4-μm microbeads. Arrows indicate flow direction. Scale bars represent the length of 1 mm.

FIGS. 16(A-C) illustrate scanning Electron Microscopy images of normal and HgCl₂-treated RBCs at 6500× magnification. (A) Non-treated RBCs (control) maintained their intact biconcave morphology. Hg²⁺-exposed RBCs underwent morphology changes and turned into echinocytes (indicated by arrows) after 3 h at 37° C. incubation with 5 μM (B) and 50 μM (C) mercuric ion. Scale bars represent a length of 5 μm.

FIGS. 17(A-D) illustrate validation of oxygen diffusion within the microchannel. (A) Tris(4,7-diphenyl-1,10-phenanthroline)ruthenium(II) dichloride complex dissolved in ethanol was injected into the deoxygenation microsystem. Luminescent intensity was measured under illumination at 488 nm. (B) Gray-level intensity near the outlet was analyzed via image processing. Solution was perfused before 0 s while the controlled gas flow (95% N₂ and 5% CO₂) was allowed at 0 s. (C) Fluorescent image obtained at 0 s. (D) Fluorescent image obtained at 420 s. Saturation of luminescent intensity indicated completion of gas exchange.

FIGS. 18(A-B) illustrate microvascular occlusion mediated by Plasmodium falciparum-infected RBCs. (A) Close-up view of parasitized RBCs retained by the 4-μm microcapillaries. Parasites were labeled with Hoechst stain. Scale bar represents a length of 10 μm. (B) RBC Occlusion Index generated by pf-infected RBCs is significantly greater compared to normal RBCs (p=0.012, Mann-Whitney). Data point cross bars represent the mean.

FIGS. 19(A-D) illustrate an analysis of RBC Occlusion Index change of stored RBCs as the blood storage progressed. Data of RBC occlusion index (A), hemolysis level (B), glucose consumption (C) and lactate production (D) plotted against storage course.

FIGS. 20(A-E) illustrates development of the microfluidic device for concurrent assessment of RBC adhesion and microvascular occlusion. (A) The microfluidic design targets at two cellular interactions: retention and adhesion. Inset: Human laminin is immobilized on the microchannel surface and adhesion receptor, BCAM/LU, on sickle RBC membrane in targeted. Laminin is immobilized on (B) PDMS surface through cross-linkers MPTES and GMBS, and (C) glass surface through cross-linkers APTES and GMBS. (D) Schematics of the device layout show that a series of micropillar arrays are embedded into the microchannel, which form microcapillaries from 20 μm down to 4 μm along the flow direction. (E) A micro view of the device with blood flow is shown.

FIG. 21 illustrates representative images of RBC adhesion and microvascular occlusion. Two microscopic images at the 4 μm array and the 10 μm array are shown. At the upstream with larger microcapillaries and low shear, RBCs adhered on the microchannel wall, while at the downstream with smaller microcapillaries and high shear, RBCs obstructed the microcapillaries.

FIGS. 22(A-B) illustrate Plasma separation by fibrinogen-accelerated RBC sedimentation. (A) Visual RBC sedimentation over a period of 20 min accelerated by additional fibrinogen at four different concentrations compared to the control groups. (B) No appreciable RBC sedimentation was observed in whole blood sample in 20 min.

FIGS. 23(A-B) illustrate microfluidic device for excluding structurally stiffened RBCs. (A) This concept is based on embedded microengineered micropillars forming various micro constrictions within the channel. (Insets) Scanning electron microscopy (SEM) images at two different magnifications showing the fabricated micropillars. Scale bars represent a length of 100 μm and 10 μm, respectively. (B) A macroscale image of an assembled chip. (Insets) A macroscale image of the chip after blood sample processing. Retained RBCs within the micropillar arrays were shown in phase contrast microscope image. Scale bar represents a length of 20 μm.

FIGS. 24(A-C) illustrate microfluidic filtration of remaining RBCs. Phase contrast microscope images showed the cell density within the separated plasma before (A) and after (B) the microfluidic filtration in hemacytometer. (C) A comparison of the numbers of cells quantified within the separated plasma before and after the microfluidic filtration.

### DETAILED DESCRIPTION

To facilitate the understanding of this invention, a number of terms are defined below. Terms defined herein have meanings as commonly understood by a person of ordinary skill in the areas relevant to the present invention. Terms such as “a”, “an”, and “the” are not intended to refer to only a singular entity but also plural entities and also includes the general class of which a specific example may be used for illustration. The terminology herein is used to describe specific aspects of the invention, but their usage does not delimit the invention, except as outlined in the claims.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

As used herein, the term “about” or “approximately” refers to a quantity, level, value, number, frequency, percentage, dimension, size, amount, weight or length that varies by as much as 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation. “Optional” or “optionally” means that the subsequently described circumstance may or may not occur, so that the description includes instances where the circumstance occurs and instances where it does not.

The term “microchannels” as used herein refer to pathways through a medium, e.g., silicon, that allow for movement of liquids and gasses. Microchannels can therefore connect other components, i.e., keep components “in fluid communication.” While it is not intended that the present application be limited by precise dimensions of the channels, illustrative ranges for channels are as follows: the channels can be between 0.1 and 100 μm in depth (e.g., 50 μm) and between 50 and 10,000 μm in width (e.g., 400 μm). The channel length can be between 1 mm and 100 mm (e.g., about 27 mm).

The term “microfabricated”, “micromachined”, and/or “micromanufactured” as used herein means to build, construct, assemble or create a device on a small scale, e.g., where components have micron size dimensions or microscale.

The term “polymer” as used herein refers to a substance formed from two or more molecules of the same substance. Polymers may also be linear polymers in which the molecules align predominately in chains parallel or nearly parallel to each other. In a non-linear polymer, the parallel alignment of molecules is not required.

The term “lensless image” or “lensless mobile imaging system” as used herein refers to an optical configuration that collects an image based upon electronic signals as opposed to light waves. For example, a lensless image may be formed by excitation of a charged coupled device (CCD) sensor by emissions from a light emitting diode.

The term “charge-coupled device (CCD)” as used herein refers to a device for the movement of electrical charge, usually from within the device to an area where the charge can be manipulated, for example, a conversion into a digital value. A CCD provides digital imaging when using a CCD image sensor where pixels are represented by p-doped MOS capacitors.

The term “symptom” as used herein refers to any subjective or objective evidence of disease or physical disturbance observed by the patient. For example, subjective evidence is usually based upon patient self-reporting and may include, but is not limited to, pain, headache, visual disturbances, nausea, and/or vomiting. Alternatively, objective evidence is usually a result of medical testing including, but not limited to, body temperature, complete blood count, lipid panels, thyroid panels, blood pressure, heart rate, electrocardiogram, tissue, and/or body imaging scans.

The term “disease” or “medical condition”, as used herein, refers to any impairment of the normal state of the living animal that interrupts or modifies the performance of the vital functions. Typically manifested by distinguishing signs and symptoms, it is usually a response to: i) environmental factors (as malnutrition, industrial hazards or climate); ii) specific infective agents (as worms, bacteria or viruses); iii) inherent defects of the organism (as genetic anomalies); and/or iv) combinations of these factors.

The term “patient” or “subject” as used herein is a human or animal and need not be hospitalized. For example, out-patients, persons in nursing homes are “patients.” A patient may comprise any age of a human or non-human animal and therefore includes both adult and juveniles, i.e., children. It is not intended that the term “patient” connote a need for medical treatment and, thus, a patient may voluntarily or involuntarily be part of experimentation whether clinical or in support of basic science studies.

The term “functionalized” or “chemically functionalized” as used herein means the addition of functional groups onto the surface of a material by chemical reaction(s). As will be readily appreciated by a person skilled in the art, functionalization can be employed for surface modification of materials in order to achieve desired surface properties, such as biocompatibility, wettability, and so on. Similarly, the term “biofunctionalization,” “biofunctionalized,” or the like, as used herein, means modification of the surface of a material to have desired biological function, which will be readily appreciated by a person of skill in the related art, such as bioengineering.

The term “sample” as used herein is used in its broadest sense and includes environmental and biological samples. Environmental samples include material from the environment such as soil and water. Biological samples may be animal, including, human, fluid, e.g., blood, plasma, and serum; solid, e.g., stool; tissue; liquid foods, e.g., milk; and solid foods, e.g., vegetables. A biological sample may comprise a cell, tissue extract, body fluid, chromosomes or extrachromosomal elements isolated from a cell, genomic DNA (in solution or bound to a solid support such as for Southern blot analysis), RNA (in solution or bound to a solid support such as for Northern blot analysis), cDNA (in solution or bound to a solid support) and the like.

The terms “capturing agent”, “bioaffinity ligand”, “binding component”, “ligand” or “receptor” as used herein may be any of a large number of different molecules, biological cells or aggregates, and the terms are used interchangeably. Each capturing agent may be immobilized on a solid substrate and binds to an analyte being detected. Proteins, polypeptides, peptides, nucleic acids (nucleotides, oligonucleotides and polynucleotides), antibodies, ligands, saccharides, polysaccharides, microorganisms such as bacteria, fungi, and viruses, receptors, antibiotics, test compounds (particularly those produced by combinatorial chemistry), plant and animal cells organdies or fractions of each and other biological entities may each be a capturing agent. Each, in turn, also may be considered as analytes if same bind to a capturing agent.

The terms “bind” or “adhere” as used herein include any physical attachment or close association, which may be permanent or temporary. Generally, an interaction of hydrogen bonding, hydrophobic forces, van der Waals forces, covalent and ionic bonding etc., facilitates physical attachment between the capturing agent and the analyte being measured. The “binding” interaction may be brief as in the situation where binding causes a chemical reaction to occur. That is typical when the binding component is an enzyme and the analyte is a substrate for the enzyme. Reactions resulting from contact between the capturing agent and the analyte are also within the definition of binding for the purposes of this application.

The term, “substrate” as used herein refers to surfaces as well as solid phases, which may include a microchannel. In some cases, the substrate is solid and may comprise PDMS. A substrate may also include components including, but not limited to, glass, silicon, quartz, plastic or any other composition capable of supporting photolithography.

The term, “photolithography”, “optical lithography” or “UV lithography” as used herein refers to a process used in microfabrication to pattern parts of a thin film or the bulk of a substrate. It uses light to transfer a geometric pattern from a photomask to a light-sensitive chemical “photoresist” or simply “resist,” on the substrate. A series of chemical treatments then either engraves the exposure pattern into or enables deposition of a new material in the desired pattern upon, the material underneath the photo resist. For example, in complex integrated circuits, a modern CMOS wafer will go through the photolithographic cycle up to 50 times.

Embodiments described herein relate to a microfluidic device and system for measuring cell deformability, occlusion, and/or adhesion both at the single cell level and in bulk, and particularly relates to a microfluidic device and system for assessing red blood cell (RBC) deformability and microvascular occlusion rate associated with RBC deformability, occlusion, and adhesion. The microfluidic device described herein can mimic architectural features associated with capillary beds of vasculature or microvasculature of a subject. For example, microfluidic device can mimic 20-μm to 4-μm narrow blood vessels to replicate the specific deformation of RBCs when traversing the microvascular bed through capillaries and small venules. Moreover, the microfluidic device described herein can enable repeated mechanical deformation cycles of RBCs in a wide range to mimic RBCs passing through numerous capillaries during their lifetime. Additionally, to test clinical samples with near-physiological hematocrit levels, the microfluidic device can have wide openings mimicking the arteriovenous anastomoses, which enable full utilization of microvasculature features and prevent complete flow obstruction.

In some embodiments, the microfluidic device can include a plurality of micropillar arrays within a microchannel that define a plurality of microcapillaries that mimic capillary networks of microvasculature of a subject. These microcapillaries can be engineered to retain RBCs with impaired deformability, such that more abnormal RBCs will occlude wide upstream microcapillaries, while those with moderate impairment will occlude finer downstream microcapillaries within the microchannel. The microchannel can also be designed with two wide side or outer openings or passages to mimic arteriovenous anastomoses, which act as shunts in the capillary bed in vivo. These side anastomoses can prevent complete blockade of flow in the microchannel, and enable testing of clinical blood samples with near-physiological hematocrit levels.

Advantageously, RBCs perfused through the microfluidic device experience a wide spectrum of deformations when crossing microcapillaries with different sizes, which recapitulates a more physiologically relevant microenvironment. Additionally, the microfluidic device can examine large numbers of heterogeneous RBCs since the embedded micropillar arrays recapitulate large numbers of microcapillaries with various dimensions, enabling the simultaneous deformability analysis of bulk RBCs at a single-cell level. The visual quantification of occlusions induced by poorly deformable RBCs, which are retained by the microcapillaries within each micropillar array, and the resulting analysis, make the assessment of overall RBC deformability and associated microvascular occlusion possible. Moreover, the microfluidic device can be used to assess very small changes in RBC deformability, under both normoxic (ambient air) and hypoxic conditions, to assess pathologically impaired RBCs in blood. The assessment of pathologically impaired RBCs can be used to assess microvascular health and function of a subject and determine the subject's increased risk of vaso-occlusive crises (VOC) in a range of microcirculatory diseases.

In some examples, the microfluidic device or system can measure or determine deformability and/or adherence of red blood cells (RBCs) of a subject. This can be used, for example, to monitor disease severity, treatment response, treatment effectiveness in a clinically meaningful way.

In one example, the cells are derived from whole blood of a subject, and the microfluidic system can be used to identify and/or measure the efficacy of therapeutic agents in treating various disorders by measuring the deformability, adherence, and/or occlusion properties of the cells under physiological flow or physiological relevant shear stress conditions and normoxia or hypoxia conditions.

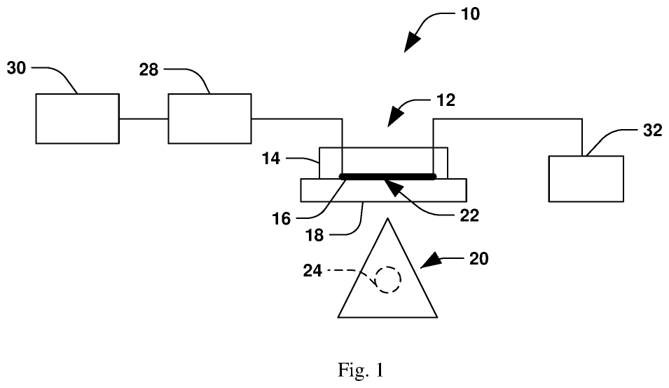

FIG. 1 illustrates a schematic view of a microfluidic system 10 in accordance with an embodiment described herein. The microfluidic system 10 includes a microfluidic device or occlusionchip 12 that has a housing 14 and at least one microchannel 16 in the housing 14 that permits fluid sample flow through the housing 14 along a length of the microchannel 16 from a first end to a second end of the microchannel. The microchannel 16 includes at least one cell occlusion region 22 within the microchannel 16. The fluidics associated the microchannels 16 can be arranged such that flow through each microchannel(s) travels in the same direction, or in opposite directions. When a microfluidic device 10 contains at least two microchannels and the fluidics associated the channels are arranged such that flow through each microchannel(s) travels in the same direction, the microchannels are typically either partially fluidically isolated or fluidically isolated. Microchannels that are “fluidically isolated” are configured and designed such that there is no fluid exchanged directly between the microchannels. Microchannels that are “partially fluidically isolated” are configured and designed such that there is partial (e.g., incidental) fluid exchanged directly between the channels.

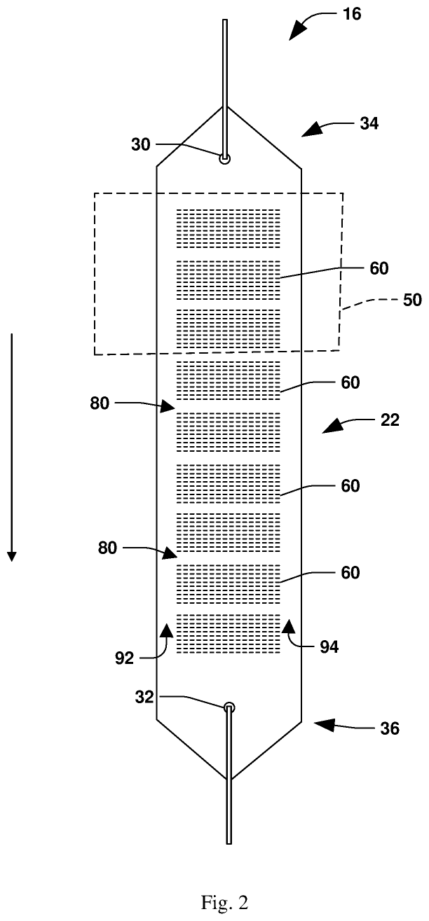

Referring to FIG. 2. the microchannel 16 is fluidly connected to an inlet port 30 at a first end 34 and an outlet port 32 at a second end 36. The inlet port 30 allows fluid to from through the microchannel 16 from the first end 34 to the second end 36 and out the outlet port 32. The fluid direction is indicated by the arrow. Although FIG. 2 depicts one microchannel, the microfluidic device 12 can include more microchannels.

The housing 14 including the at least one microchannel 16 can further contain a substantially planar transparent wall 18 that defines a surface of at least one of the microchannels 16. This substantially planar transparent wall 18, which can be, for example, glass or plastic, permits observation into the microchannel 16 by an imaging system 20 (e.g., microscopy) so that at least one measurement of each cell that passes through the cell occlusion region 22 of one of the microfluidic channels 16 can be obtained. In one example, the transparent wall has a thickness of 0.05 mm to 1 mm. In some cases, the transparent wall 18 may be a microscope cover slip, or similar component. Microscope coverslips are widely available in several standard thicknesses that are identified by numbers, as follows: No. 0-0.085 to 0.13 mm thick, No. 1-0.13 to 0.16 mm thick, No. 1.5-0.16 to 0.19 mm thick, No. 2-0.19 to 0.23 mm thick, No. 3-0.25 to 0.35 mm thick, No. 4-0.43 to 0.64 mm thick, any one of which may be used as a transparent wall 18, depending on the device, microscope, and detection strategy.

In some embodiments, the microchannel(s) 16 may have a depth or height in a range of 0.5 μm to 100 μm, 0.1 μm to 100 μm, 1 μm to 50 μm, 1 μm to 50 μm, 10 μm to 40 μm, 5 μm to 15 μm, 0.1 μm to 5 μm, or 2 μm to 5 μm. The microchannel(s) may have a depth or height of up to 0.5 μm, 1 μm, 1.5 μm, 2.0 μm, 2.5 μm, 3.0 μm, 3.5 μm, 4.0 μm, 4.5 μm, 5.0 μm, 5.5 μm, 6.0 μm, 6.5 μm, 7.0 μm, 7.5 μm, 8.0 μm, 8.5 μm, 9.0 μm, 9.5 μm, 10 μm, 20 μm, 30 μm, 40 μm, 50 μm, 75 μm, 100 μm, or more.

In some embodiments, the at least one microchannel 16 can have a cross-sectional area, perpendicular to the flow direction, of 500 μm², 600 μm², 700 μm², 800 μm², 900 μm², 1000 μm², 2000 μm², 3000 μm², 4000 μm², 5000 μm², 6000 μm², 7000 μm², 8000 μm², 9000 μm², 10,000 μm² or more.

The microfluidic device 10 may be designed and configured to produce any of a variety of different shear rates (e.g., up to 100 dynes/cm²). For example, the microfluidic device 10 may be designed and configured to produce a shear rate in a range of 0.1 dynes/cm² to 10 dynes/cm², 0.5 dynes/cm² to 5 dynes/cm², 0.5 dynes/cm² to 2 dynes/cm², 0.6 dynes/cm² to 1.5 dynes/cm², 0.7 dynes/cm² to 1.3 dynes/cm², 0.8 dynes/cm² to 1.2 dynes/cm², or 0.9 dynes/cm² to 1.1 dynes/cm², or 1 dynes/cm².

Referring to FIG. 2, the occlusion region 22 of the microchannel 16 can include a plurality of micropillar arrays 40 provided along the length of the microchannel 16. The micropillar arrays 40 can be arranged in series such that a flow path through one micropillar array 40 is parallel with a flow path through the other micropillar arrays 40 in series.

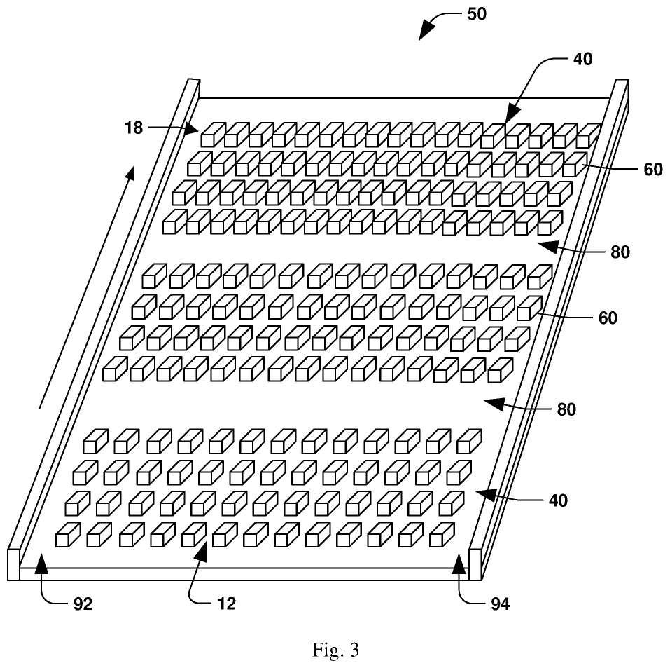

FIG. 3 is a schematic illustration of a cut-out section 50 of the microchannel 16 of FIG. 2. The cut-out section 50 illustrate three micropillar arrays 40 provided in the occlusion region 22 of the microchannel 16. Each micropillar array 40 includes a plurality of micropillars 60 arranged in substantially parallel rows that extend perpendicular to the direction of fluid flow, which is illustrated by the arrow. While FIG. 3 shows the micropillars 60 have a shape that is substantially rectangular or box like, it will be appreciated the micropillars 60 of the micropillar arrays 40 can have any of variety of shapes, including, for example, polygonal (e.g., triangular, hexagonal), curvilinear or circular shapes.

In some embodiments, the micropillars 60 of each micropillar array 40 and optionally all the micropillars 60 of the micropillar arrays 40 can have the same shape and size. For example, the micropillars 60 of each micropillar array 40 can have a substantially rectangular cross section and box like shape. In some embodiments, each of the micropillars 60 of the micropillar array 40 can have a width (w), which is parallel to the width of the microchannel, of up to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or more, a height (h), which is parallel to the height of the microchannel, of up to about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or more, and a length, which is parallel to the length of the microchannel, of up to about 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, 21 μm, 22 μm, 23 μm, 24 μm, 25 μm, 26 μm, 27 μm, 28 μm, 29 μm, or 30 μm or more. In one example, each micropillar 60 of the micropillar array 40 can have a width of about 10 μm, a height of about 12 μm, and a length of about 20 μm.

The micropillars 60 of each micropillar array 40 can be arranged in evenly or uniformly spaced rows 62. The rows 62 of micropillars 60 can extend perpendicular to fluid flow through the microchannel 16. The rows 62 of micropillars 60 in each micropillar array 40 can be substantially parallel to one another. The number of rows 62 of micropillars 60 in each micropillar array 40 can be the same or vary.

In some embodiments, each micropillar array can include at least 3, 4, 5, 6, 7, 8, 9, 10, or more rows of micropillars 60. In one example, each micropillar array 40 can include about 5 to about 10 rows that are even spaced from each other by, for example, about 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, or 20 μm, 21 μm, 22 μm, 23 μm, 24 μm, 25 μm, 26 μm, 27 μm, 28 μm, 29 μm, or 30 μm or more.

The number of micropillars 60 in each row 62 can vary depending on the width of the microchannel 16, the width of the micropillars 60, and the spacing between respective micropillars 60. In some embodiments, at least 10, 11, 12, 13, 14, 15, 16, 17, 18, 19, 20, 21, 22, 23, 24, 25, 26, 27, 28, 29, 30, 31, 32, 33, 34, 35, 36, 27, 38, 39, 40, 41, 42, 43, 44, 45, 46, 47, 48, 49, 50 or more uniformly spaced micropillars 60 can be arranged in row of each micropillar array 40.

The micropillars 60 in each row 62 can be evenly or uniformly spaced from each other such that the distance between each micropillar 60 in a respective row 62 of each micropillar array 40 is substantially the same. The distance between each micropillar in a respective row will vary depending upon the micropillar array and can be, for example, about 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm or more.

Successive micropillar arrays 40 in path of fluid flow through the microchannel 16 have decreasing distances between the micropillars 60 in the rows 62. For example, micropillars in a first micropillar array at the first end of the microchannel can be separated from each other in a row by about 20 μm, micropillars in a successive second micropillar array downstream of the first micropillar array can be separated from each other in a row by about 18 μm, micropillars in a successive third micropillar array downstream of the second micropillar array can be separated from each other in a row by about 16 μm, micropillars in a successive fourth micropillar array downstream of the third micropillar array can be separated from each other in a row by about 14 μm, micropillars in a successive fifth micropillar array downstream of the fourth micropillar array can be separated from each other in a row by about 12 μm, micropillars in a successive sixth micropillar array downstream of the fifth micropillar array can be separated from each other in a row by about 10 μm, micropillars in a successive seventh micropillar array downstream of the sixth micropillar array can be separated from each other in a row by about 8 μm, micropillars in a successive eighth micropillar array downstream of the seventh micropillar array can be separated from each other in a row by about 6 μm, and micropillars in a successive ninth micropillar array downstream of the eighth micropillar array can be separated from each other in a row by about 4 μm.

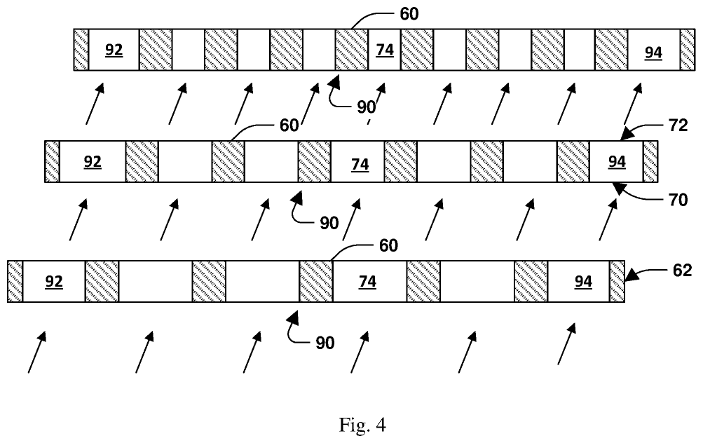

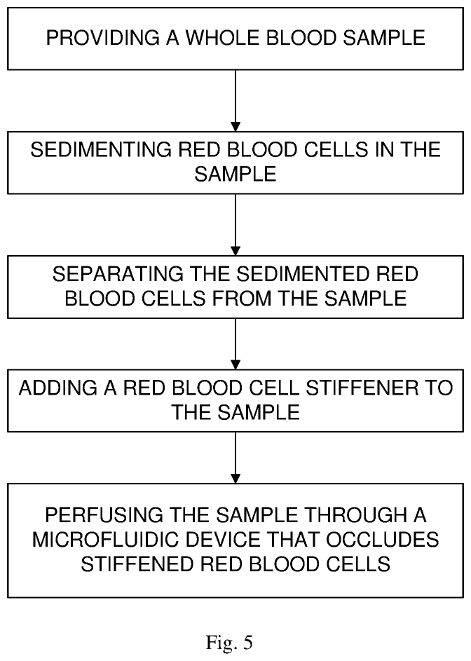

Referring to FIG. 4 which is a schematic illustration of cross-section of flow through a single row of micropillars of successive micropillar arrays, the micropillars 60 of the micropillar arrays 40 can extend from a substantially planar lower surface 70 to a substantially planar upper surface 74 of the microchannel 16 such that the micropillars 60 of each row of the micropillar array 40 define a plurality of microcapillaries 74 having a cross sectional area that is defined by the width and height of the micropillars in a respective row of a micropillar array. The plurality of microcapillaries 74 defined by each micropillar array 40 can be arranged in series such that a flow path through one microcapillary 74 is parallel with a flow path through the other microcapillaries 74. The microcapillaries 74 defined by the micropillars in each row 62 can have substantially the same width, height, and cross-sectional area.

Referring to FIGS. 3 and 4, the width and/or cross sectional area of the microcapillaries 74 defined by each micropillar array 40 decreases in a direction of fluid flow through the microchannel 16 in accordance with decreasing distance between each micropillar 74 in each micropillar array 40. The decreasing width and/or cross sectional area of the microcapillaries 74 of the successive micropillar arrays 40 can mimic capillary networks of a subject and be engineered to retain cells, such RBCs, with impaired deformability so that more abnormal RBCs will occlude wide upstream microcapillaries 74, while those with moderate impairment will occlude finer downstream microcapillaries 74 within the microchannel 16.

Each of the microcapillaries 74 defined by a respective micropillar array 40 can have a substantially uniform width, height, and cross-sectional area perpendicular to the flow direction through the microcapillaries 74. The micropillars 60 in successive rows 62 of each micropillar array 40 can be aligned directly behind or offset from the micropillars 60 in a preceding row 62 such that microcapillaries 74 defined by a successive or preceding row 62 are offset perpendicular to the direction of fluid flow.

In some embodiments, a micropillar array 40 at the first end 34 defines a plurality of microcapillaries that can each have a width of about 18 μm to about 22 μm (e.g., about 20 μm). Each successive micropillar array in the direction of fluid flow through the microchannel can define a plurality of microcapillaries that can each have a width at least about 1%, about 5%, about 10%, about 15%, about 20%, about 25%, about 30%, about 35%, about 40%, about 45%, about 50%, about 55%, or about 60% less (e.g., about 5% to about 50% less) than a plurality of microcapillaries defined by a preceding micropillar array. For example, microcapillaries defined by a successive second micropillar arrays downstream of the micropillar array at first end can each have a width of about 20 μm, about 19 μm, about 18 μm, about 17 μm, about 16 μm, about 15 μm, about 14 μm, about 13 μm, about 12 μm, about 11 μm, about 10 μm, about 9 μm, about 7 μm, about 6 μm, about 5 μm, about 4 μm, about 3 μm, about 2 μm, or about 1 μm or less.

In some embodiments, a micropillar array 40 at the first end 34 defines a plurality of microcapillaries that can each have a cross sectional area of about 200 μm² to about 250 μm² (e.g., about 230 μm² to about 250 μm²). Each successive micropillar array in the direction of fluid flow through the microchannel can define a plurality of microcapillaries that can each have a cross sectional area at least about 1%, about 5%, about 10%, about 15%, about 20%, about 25%, about 30%, about 35%, about 40%, about 45%, about 50%, about 55%, or about 60% less (e.g., about 5% to about 50% less) than a plurality of microcapillaries defined by a preceding micropillar array. For example, microcapillaries defined by a successive second micropillar arrays downstream of the micropillar array at first end can each have successive cross sectional areas of about 220 μm² to about 230 μm², about 210 μm² to about 220 μm², about 200 μm² to about 210 μm², about 190 μm² to about 200 μm², about 180 μm² to about 190 μm², about 170 μm² to about 190 μm², about 160 μm² to about 170 μm², about 150 μm² to about 160 μm², about 140 μm² to about 150 μm², about 130 μm² to about 140 μm², about 120 μm² to about 130 μm², about 110 μm² to about 120 μm², about 100 μm² to about 110 μm², about 90 μm² to about 100 μm², about 80 μm² to about 90 μm², about 70 μm² to about 80 μm², about 60 μm² to about 70 μm², about 50 μm² to about 60 μm², about 40 μm² to about 50 μm², or about 30 μm² to about 40 μm²

In other embodiments, a micropillar array 40 at the second end 36 defines a plurality of microcapillaries that can each have a width of about 3 μm to about 5 μm (e.g., about 4 μm). Each preceding micropillar array opposite the direction of fluid flow through the microchannel from the second end 36 can define a plurality of microcapillaries that can each have a width at least about 1%, about 5%, about 10%, about 15%, about 20%, about 25%, about 30%, about 35%, about 40%, about 45%, about 50%, about 55%, or about 60% greater (e.g., about 5% to about 50% greater) than a plurality of microcapillaries defined by a preceding micropillar array. For example, microcapillaries defined by a preceding micropillar array upstream of the micropillar array at the second end can each have a width of about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm or more.

In other embodiments, the microchannel 16 includes a micropillar array 40 at the second end 36 that defines a plurality of microcapillaries that can each have a cross sectional area of about 40 μm² to about 50 μm². Each preceding micropillar array opposite the direction of fluid flow through the microchannel from the second end 36 can define a plurality of microcapillaries that can each have a cross sectional area at least about 1%, about 5%, about 10%, about 15%, about 20%, about 25%, about 30%, about 35%, about 40%, about 45%, about 50%, about 55%, or about 60% greater (e.g., about 5% to about 50% greater) than a plurality of microcapillaries defined by a preceding micropillar array. For example, microcapillaries defined by a preceding micropillar array upstream of the micropillar array at the second end can each have a cross sectional area of about 50 μm² to about 60 μm², about 60 μm² to about 70 μm², about 70 μm² to about 80 μm², about 80 μm² to about 90 μm², about 90 μm² to about 100 μm², about 100 μm² to about 110 μm², about 110 μm² to about 120 μm², about 120 μm² to about 130 μm², about 130 μm² to about 140 μm², about 140 μm² to about 150 μm², about 160 μm² to about 170 μm², about 170 μm² to about 180 μm², about 180 μm² to about 190 μm², about 190 μm² to about 200 μm², about 200 μm² to about 210 μm², about 210 μm² to about 220 μm², about 220 μm² to about 230 μm², about 230 μm² to about 240 μm², or about 240 μm² to about 250 μm².

In some embodiments, the width, height, and cross sectional area of the microcapillaries 74 defined by at least one of the plurality of micropillar arrays 40 permits passage of healthy cells in a fluid sample perfused through the microchannel but occludes cells with impaired deformability. The cell can be blood cells, such as red blood cells.

In other embodiments, the width, height, and cross sectional area of the plurality of microcapillaries 74 at the second end 36 of the microchannel 16 can occlude cells in a fluid sample perfused through the at least one microchannel 16.

In some embodiments, as illustrated in FIGS. 2 and 3, successive micropillar arrays 40 can be separated from each other in the microchannel by a gap region 80, which is free of micropillars 60. In one example, this gap region 80 is of a length that allows cells, such as RBCs, in the fluid sample to recover, at least partially, their shape after passing through the microcapillaries of a respective micropillar array. In another example, the gap region is of a length that does not allow one or more cells in the fluid sample to recover its shape after passing through the microcapillaries of a respective micropillar array.

The gap region 80 may have a length (e.g., distance between respective micropillar arrays) of up to 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more. For example, the gap region may have a length in a range of 25 μm to 100 μm, 50 μm to 125 μm, 75 μm to 150 μm, or 100 μm to 200 μm.

In some embodiments, each of the micropillar arrays 40 can be arranged in an inner portion 90 of the microchannel 16 that extends the length of the microchannel 16. The microchannel 16 can include two parallel outer or side passages 92 and 94 on opposite sides of the inner portion 90 that extend the length of the microchannel 16. The outer passages 92 and 94 are designed to mimic arteriovenous anastomoses which act as shunts in the capillary bed in vivo. These outer passages 92 and 94 can prevent complete blockade of flow in the microchannel, and enable testing of clinical blood samples with near-physiological hematocrit levels. The outer passages 92 and 94 can be in fluid communication with the plurality of microcapillaries 74 defined by the plurality micropillar arrays 40. The outer passages can have cross sectional areas that permit cells in a fluid sample to flow through the microchannel without being occluded and/or obstructed. In some embodiment the outer passages 92 and 94 can have widths of 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 1 mm or more. For example, the outer passages 92 and 94 may have widths in a range of 25 μm to 100 μm, 50 μm to 125 μm, 75 μm to 150 μm, or 100 μm to 200 μm

Optionally, where the microfluidic device 12 is designed to occlude all cells that pass through the microchannel, the outer passages can be omitted so that the micropillar arrays 40 extend to opposite walls that define the width of the microchannel. In this instance, fluid passing through the microchannel will not be able to bypass the microcapillaries defined by the micropillar arrays.

In some embodiments, the microfluidic system 10 can simulate physiologically relevant shear gradients (e.g., 0.5 dynes/cm² to about 2 dynes/cm²) of microcirculatory blood flow at a constant single volumetric flow rate. Using this system, shear-dependent adhesion, occlusion, and deformability of cells, for example, RBCs and WBCs, from subjects with disorders, such as SCD can be investigated. It was shown that shear dependent adhesion of cells, such as RBCs and WBCs, exhibit a heterogeneous behavior based on adhesion type and cell deformability in a microfluidic flow model, which correlates clinically with inflammatory markers and iron overload in patients with SCD. This revealed the complex dynamic interactions between RBC-mediated microcirculatory occlusion and clinical outcomes in SCD. These interactions may also be relevant to other microcirculatory disorders

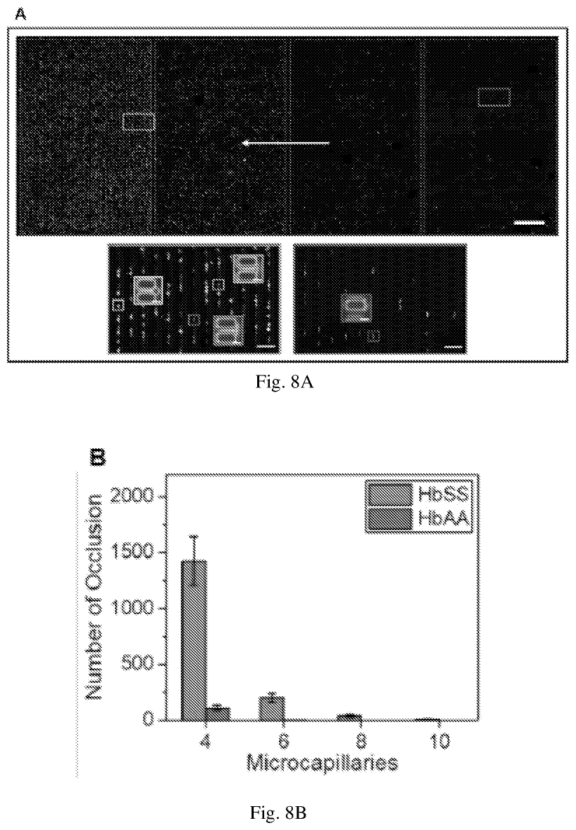

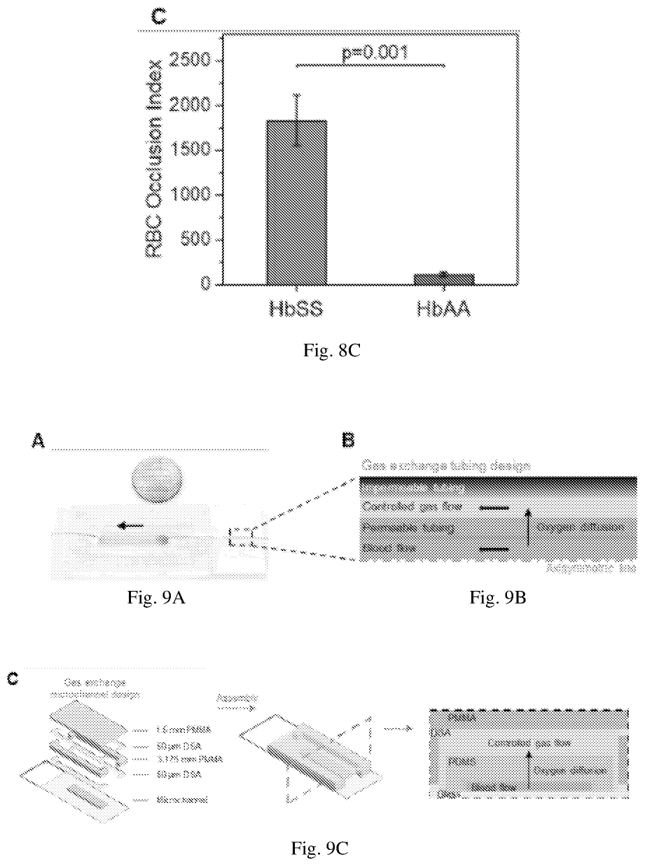

In some embodiments, as illustrated in FIG. 8C, the microfluidic device can include a multilayer structure formed of a base layer, a microchannel, intermediate layers, and a cover layer. The microchannel includes the occlusion region. Referring to FIG. 2, a first end 34 of the microchannel 16 is aligned with a corresponding inlet port 30. A second end 36 of the microchannel 16 is aligned with a corresponding outlet port 32. This creates a flow channel from an inlet port 30 to the corresponding outlet 32 port via the microchannel 16. The microchannel 16 can also extend slightly beyond its respective inlet port 30 and outlet port 32. The microchannel is sized to accept volumes, e.g., μL or mL, of the fluid sample containing cells to be occluded, adhered, or captured in the occlusion region.

Referring again to FIG. 8C, the base layer provides structural support to the microchannel and is formed of a sufficiently rigid, optically transparent, and gas impermeable material, such as poly(methyl nethaciylate) (PMMA) or glass. The base layer can have a suitable thickness, for example of about 0.1 mm to about 2 mm, or about 1.6 mm, determined by manufacturing and assembly restrictions.

The cover layer contains the inlet ports and outlet ports used to feed the sample in/out of the microchannel. The cover layer thickness can be about 1 mm to about 10 mm, for example, about 3.6 mm, and is determined by the integration and assembly requirements. The inlet and outlet port diameters can be about 0.3 mm to about 3 mm, for example about 1 mm. The lower size limit is determined by the manufacturing restrictions. The upper size limit is determined by the desired flow conditions of sample through the channel. In another example (not shown), a laser cutter can be used to cut a larger piece of PMMA into a desired size for the microfluidic device and to cut holes for the inlet ports and the outlet ports.

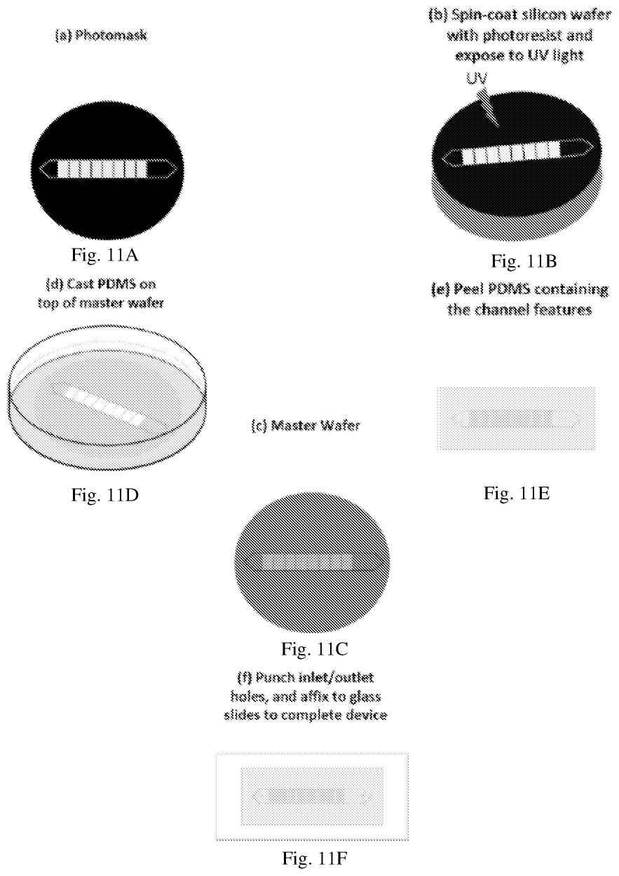

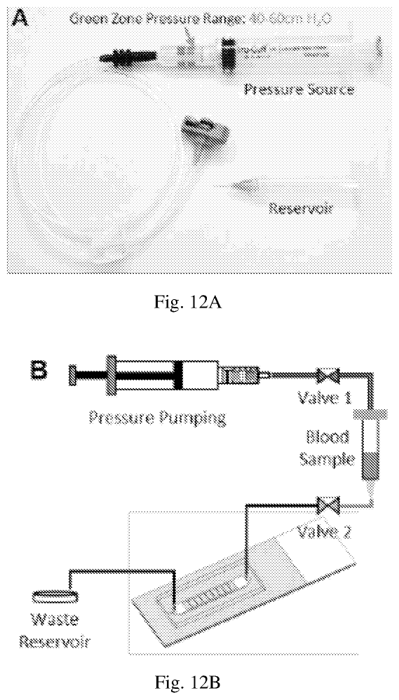

The microchannel can include a plurality of micropillar arrays that can be fabricated through photolithography. By way of example, as illustrated in FIG. 12, a photomask with designed microchannel features can be used to pattern a silicon wafer. The wafer can be spin-coated with a negative photoresist layer, soft-baked, and exposed to UV light under the photomask. After post-exposure baking, developing and hard baking, the master wafer is completed. The wafer can be covered with PDMS and then cured. The cured PDMS block can then peeled-off from the master wafer, and two holes punched as the inlet and the outlet to define the microchannel. After cleaning, the PDMS block, which forms the microchannel and micropillar arrays, can be bonded to the base layer.

The intermediate layers can adhered to the base layer around the microchannel after the microchannel is placed on the base layer. The cover layer, which can have the same lateral dimensions as the base layer and the intermediate layer, can be adhered onto the exposed side of the intermediate layer, thereby enclosing the microchannel. In the examples depicted, the microfluidic device is oriented such that the cover layer is on top. Alternatively, the microfluidic device can be oriented such that the cover layer is on the bottom (not shown).

In some embodiments, at least one surface of the cell occlusion region 22 of the microchannel 16, including the surface of the micropillars, can be functionalized with at least one capturing agent or bioaffinity ligand that captures or adheres a cell of interest to a surface of the microchannel when a sample fluid containing cells is passed or perfused through the at least one microchannel. If the housing includes multiple microchannels, each microchannel can be functionalized with a different capturing agent to adhere different cells of interest thereto. In any case, each microchannel is configured to receive and provide cell adhesion analysis of a microvolume fluid sample.

In some embodiments, the capturing agents can include, for example, bioaffinity ligands or adhesion molecules that are associated with an activated phenotype in a hematological or circulatory disease or disorder, such as SCD. Such bioaffinity ligands or adhesion molecules can include, for example, at least one of laminin, fibronectin, selectins, such as E-Selectin, P-Selectin, or L-selectin, intracellular adhesion molecule 1 (ICAM-1), or vascular cellular adhesion molecule 1 (VCAM-1). Laminin, fibronectin, E-Selectin, P-Selectin, L-selectin, ICAM-1, or VCAM-1 can adhere to cells, such as WBCs and/or RBCs, and be used to detect and/or measure WBC and/or RBC adherence under physiological relevant shear stress and normoxic and hypoxic conditions.

The capturing agent or bioaffinity ligand can be adhered to, functionalized or chemically functionalized to the at least one surface of the cell occlusion region of the microchannel. The bioaffinity ligands may be functionalized to the at least one surface of the cell occlusion region covalently or non-covalently. A linker can be used to provide covalent attachment of a bioaffinity ligand to the surface of the cell occlusion region. The linker can be a linker that can be used to link a variety of entities.

In some examples, the linker may be a homo-bifunctional linker or a hetero-bifunctional linker, depending upon the nature of the molecules to be conjugated. Homo-bifunctional linkers have two identical reactive groups. Hetero-bifunctional linkers have two different reactive groups. Various types of commercially available linkers are reactive with one or more of the following groups: primary amines, secondary amines, sulphydryls, carboxyls, carbonyls and carbohydrates. Examples of amine-specific linkers are bis(sulfosuccinimidyl) suberate, bis[2-(succinimidooxycarbonyloxy)ethyl]sulfone, disuccinimidyl suberate, disuccinimidyl tartarate, dimethyl adipimate 2HCl, dimethyl pimelimidate 2HCl, dimethyl suberimidate HCl, ethylene glycolbis-[succinimidyl-[succinate]], dithiolbis(succinimidyl propionate), and 3,3′-dithiobis(sulfosuccinimidylpropionate). Linkers reactive with sulfhydryl groups include bismaleimidohexane, 1,4-di-[3′-(2′-pyridyldithio)-propionamido)]butane, 1-[p-azidosalicylamido]-4-[iodoacetamido]butane, and N-[4-(p-azidosalicylamido)butyl]-3′-[2′-pyridyldithio]propionamide. Linkers preferentially reactive with carbohydrates include azidobenzoyl hydrazine. Linkers preferentially reactive with carboxyl groups include 4-[p-azidosalicylamido]butylamine.

Heterobifunctional linkers that react with amines and sulfhydryls include N-succinimidyl-3-[2-pyridyldithio]propionate, succinimidyl[4-iodoacetyl]aminobenzoate, succinimidyl 4-[N-maleimidomethyl]cyclohexane-1-carboxylate, m-maleimidobenzoyl-N-hydroxysuccinimide ester, sulfosuccinimidyl 6-[3-[2-pyridyldithio]propionamido]hexanoate, and sulfosuccinimidyl 4-[N-maleimidomethyl]cyclohexane-1-carboxylate. Heterobifunctional linkers that react with carboxyl and amine groups include 1-ethyl-3-[3-dimethylaminopropyl]-carbodiimide hydrochloride. Heterobifunctional linkers that react with carbohydrates and sulfhydryls include 4-[N-maleimidomethyl]-cyclohexane-1-carboxylhydrazide HCl, 4-(4-N-maleimidophenyl)-butyric acid hydrazide. 2HCl, and 3-[2-pyridyldithio]propionyl hydrazide.

In some embodiments, a surface layer of 3-aminopropyl triethoxy silane (ATES) and/or (3-mercaptopropyl)trimethoxysilane (MTPMS) can be initially applied to surfaces of the microchannel followed by incubation with N-γ-maleimidobutyryl-oxysuccinimide ester (GMBS) to functionalize the bioaffinity ligand or capturing agent to the surfaces.

By way of example, a GMBS working solution can prepared by dissolving GMBS in DMSO and diluting with ethanol. A bioaffinity ligand described herein, such as laminin, fibronectin, E-Selectin, P-Selectin, L-selectin, ICAM-1, or VCAM-1 can be diluted with PBS to create a bioaffinity ligand working solution. The GMBS working solution can injected into the microchannels and incubated at room temperature. Following GMBS incubation, the microchannels can be washed. Next, the bioaffinity ligand working solution can injected into the microchannels and incubated at room temperature. The surface can then passivated by injecting a BSA solution incubated, thereby forming a bioaffinity ligand functionalized surface. The microchannels can be optionally rinsed with PBS before processing samples.

Alternatively, the bioaffinity ligands may be non-covalently coated onto a surface of the cell occlusion region. Non-covalent deposition of the bioaffinity ligand to the surface of the cell occlusion region may involve the use of a polymer matrix. The polymer may be naturally occurring or non-naturally occurring and may be of any type including but not limited to nucleic acid, e.g., DNA, RNA, PNA, LNA, and the like or mimics, derivatives or combinations thereof, amino acid, e.g., peptides, proteins (native or denatured), and the like or mimics, derivatives or combinations thereof, lipids, polysaccharides, and functionalized block copolymers. The bioaffinity ligand may be adsorbed onto and/or entrapped within the polymer matrix. Alternatively, the bioaffinity ligand may be covalently conjugated or crosslinked to the polymer, e.g., it may be “grafted” onto a functionalized polymer.

An example of a suitable peptide polymer is poly-lysine, e.g., poly-L-lysine. Examples of other polymers include block copolymers that comprise polyethylene glycol (PEG), polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, polyvinyl chloride, polystyrene, polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate), poly(lactide-glycolide), copolyoxalates, polycaprolactones, polyesteramides, polyorthoesters, polyhydroxybutyric acid, polyanhydrides, poly(styrene-b-isobutylene-b-styrene) (SIBS) block copolymer, ethylene vinyl acetate, poly(meth)acrylic acid, polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, albumin and other hydrophilic proteins, zein and other prolamines and hydrophobic proteins, copolymers and mixtures thereof, and chemical derivatives thereof including substitutions and/or additions of chemical groups, for example, alkyl, alkylene, hydroxylations, oxidations, and other modifications routinely made by those skilled in the art.

Referring again to FIG. 1, the microfluidic system also includes an imaging system for measuring the deformability, occlusion, and/or number of the cells of interest in the cell occlusion region and/or adhered to the at least one capturing agent of at least one microchannel when the fluid sample is passed or perfused through the microchannel under, for example, physiological relevant shear stress and normoxia or hypoxia conditions.

The imaging system 20 can detect and measure through the at least one optically transparent wall the morphology and/or quantity of occluded, adhered, and/or captured cells of interest within each microchannel and optionally the viscosity of the fluid sample. The imaging system 20 can be a lens-based imaging system, lensless imaging system, and/or mobile imaging system, e.g., cellular phone camera. The imaging system 20 can include a control unit 24, which can a include a computer readable storage unit and a processor to analyze the images of the microchannels and provide real-time feedback to a subject of the results of the image acquisition/analysis. These results, in turn, can be readily transmitted to a primary care provider and/or stored in a medical record database.

In some examples, the imaging system can be a lens-based imaging system or a lensless/mobile imaging system. In some embodiments, the lensless imaging system can be a CCD sensor and a light emitting diode. By way of example, a fluorescent microscopy camera (EXi Blue EXI-BLU-R-F-M-14-C) and an Olympus IX83 inverted, fluorescent motorized microscope with Olympus Cell Sense live-cell imaging and analysis software can be used to obtain real-time microscopic images. Olympus (20×/0.45 ph2 and 40×/0.75 ph3) long working distance objective lenses can be utilized for phase contrast imaging of cells occluded and/or adhered in the microchannels. During real-time microscope imaging and high resolution video recording at 7 fps rate, controlled fluid flow with stepwise increments can be applied until cell detachment from the microchannel surface is observed. Videos can be converted to single frame images for further processing and analysis. The cell dimensions can then analyzed by using Adobe Photoshop software (San Jose, Calif.).

In some examples, a mobile imaging and quantification algorithm can be integrated into or with the microfluidic device. The algorithm can achieve reliable and repeatable test results for data collected in all resource settings of the microfluidic device.

In other examples, the microfluidic device can be configured to cooperate with a cellular phone having imaging capabilities. In such a case, the cellular phone can be provided with or capable of obtaining image analysis algorithms/software, e.g., via an online application. Images can be recreated by the cellular phone camera software and loaded into a custom phone application that identifies occluded and/or adhered cells, such as RBCs, quantifies the number of occluded and/or adhered cells, such as RBCs, in the image, and displays the results.

The cells of interest can be blood cells obtained from the subject and the imaging system can quantify the occluded and/or adhered cells in the microchannel to measure the cell deformability and/or adherence. In other examples, the imaging system can quantify occluded and/or adhered cells in the microchannel to monitor the progression of a disease, such as SCD, of a subject from which the cells are obtained. In still other examples, the imaging system can quantify the occluded and/or adhered cells in each channel to measure the efficacy of a therapeutic treatment administered to a subject from which the cells are obtained.

In some embodiments, the imaging system 20 can be configured to provide particle image velocimetry of fluid in the microchannels. For example, the imaging system 20 can be configured to take images of fluid as it passes through an imaging field of the microchannel. These images can be sent to control unit that includes a computer readable storage medium for storing the images and a processor that include executable instructions for receiving sequential images, generating general velocity vector maps based on successive images, and generating mean flow velocity data from the velocity vector maps. The mean flow velocity data can be output from the processor to a display as raw data or as visual representation of the mean flow velocity. The mean flow velocity data or map can be correlated to viscosity of the fluid using the processor or another processor that outputs the viscosity date of the fluid as raw data or as visual depiction.

The image processing may be implemented using hardware, software or a combination thereof. When implemented in software, the software code can be executed on any suitable processor or collection of processors, whether provided in a single computer or distributed among multiple computers. Such processors may be implemented as integrated circuits, with one or more processors in an integrated circuit component. Though, a processor may be implemented using circuitry in any suitable format.

Further, it should be appreciated that a computer may be embodied in any of a number of forms, such as a rack-mounted computer, a desktop computer, a laptop computer, or a tablet computer. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible format.

Such computers may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, a computer readable medium (or multiple computer readable media) (e.g., a computer memory, one or more floppy discs, compact discs (CD), optical discs, digital video disks (DVD), magnetic tapes, flash memories, circuit configurations in Field Programmable Gate Arrays or other semiconductor devices, or other non-transitory, tangible computer storage medium) can be encoded with one or more programs that, when executed on one or more computers or other processors, perform methods that implement the various embodiments described herein. The computer readable medium or media can be transportable, such that the program or programs stored thereon can be loaded onto one or more different computers or other processors to implement various aspects described herein. As used herein, the term “non-transitory computer-readable storage medium” encompasses only a computer-readable medium that can be considered to be a manufacture (i.e., article of manufacture) or a machine.

The terms “program” or “software” are used herein in a generic sense to refer to any type of computer code or set of computer-executable instructions that can be employed to program a computer or other processor to implement various aspects as discussed above. Additionally, it should be appreciated that according to one aspect of this embodiment, one or more computer programs that when executed perform methods of described herein need not reside on a single computer or processor, but may be distributed in a modular fashion amongst a number of different computers or processors to implement various aspects herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

In some embodiments, the microfluidic system 10 can further include a reservoir 28 fluidically connected with the one or more microfluidic channels 16, and a pump 30 that perfuses fluid from the reservoir 28 through the one or more microchannels 16 to a waste or fluid collection reservoir 32. The pump 30 can designed and configured to create a pressure to create a pressure (gauge pressure) in at least one of the microchannels 16 of up to 50 Pa, 100 Pa, 200 Pa, 300 Pa, 400 Pa, 500 Pa, 600 Pa, 700 Pa, 800 Pa, 900 Pa, 1 kPa, 2 kPa, 5 kPa, 10 kPa or more. The pump 30 may be designed and configured to create a pressure (gauge pressure) in the channel in a range of 50 Pa to 200 Pa, 100 Pa to 500 Pa, 100 Pa to 800 Pa, 100 Pa to 1 kPa, 500 Pa to 5 kPa, or 500 Pa to 10 kPa.

The microfluidic system 10 may also be designed and configured to create an average fluid velocity within the channel of up to 1 μm/s, 2 μm/s, 5 μm/s, 10 μm/s, 20 μm/s, 50 μm/s, 100 μm/s, or more.

The microfluidic system 10 may be designed and configured to create an average fluid velocity within at least one microchannel 16 in a range of 1 μm/s to 5 μm/s, 1 μm/s to 10 μm/s, 1 μm/s to 20 μm/s, 1 μm/s to 50 μm/s, 10 μm/s to 100 μm/s, or 10 μm/s to 200 μm/s, for example.

In some embodiments, the reservoir 28 contains cells, such as RBCs and WBCs, suspended in a fluid, such as blood or plasma.

In some embodiments, the microfluidic system 10 can further includes a micro-gas exchanger (not shown) fluidly connected to the at least one microchannel 16 for varying the oxygen content of the fluid sample containing the cells. In one embodiment, the micro-gas exchanger can include a gas-permeable inner tube inserted within a gas-impermeable outer tube. Fluid, such as blood or synovial fluid, containing the cells of interest can be delivered through the inner tube such that the fluid exchanges gases through the permeable tubing wall with a control gas, e.g., 5% CO₂ and 95% N₂, between the tubes. The oxygen content of the fluid exiting the micro-gas exchanger is controlled to thereby control the oxygen content of the fluid delivered to the microchannel.

By way of example, the micro-gas exchanger can include concentric inner and outer tubes. The inner tube has a gas-permeable wall defining a central passage extending the entire length of the inner tube. The outer tube has a gas impermeable wall defining a central passage extending the entire length of the outer tube. An annular space is formed between the tubes. The central passage receives the fluid sample and is in fluid communication with one or more inlet ports of the microfluidic device. Each inlet port can be fluidly connected to the same micro-gas exchanger or a different micro-gas exchanger to specifically tailor the fluid delivered to each microchannel. An outlet tube is connected to each outlet port of the micro-gas exchanger. A controlled gas flow takes place in the annular space between the concentric tubes and fluid flows inside the inner tube. When the fluid sample is blood, deoxygenation of the sample occurs due to gas diffusion (5% CO₂ and 95% N₂) through the inner gas-permeable wall.

In another embodiment, the micro-gas exchanger can be integrated with the housing and the at least one microchannel 16 for varying the oxygen content of the fluid sample containing the cells. In one embodiment, the micro-gas exchanger can include a gas-permeable inner wall inserted within a gas-impermeable outer wall. Fluid, containing the cells of interest can be delivered through the microchannel such that the fluid exchanges gases through the permeable wall with a control gas, e.g., 5% CO₂ and 95% N₂, between the walls. The oxygen content of the fluid exiting the micro-gas exchanger is controlled to thereby control the oxygen content of the fluid delivered to the microchannel.

By way of example, as shown in FIG. 8, housing can include overlapping inner and outer walls. The inner wall is gas-permeable wall defines the microchannel through the housing. The outer wall is gas impermeable and defines a central passage extending the entire length of the outer wall and inner wall. A space is formed between the inner and outer walls. The microchannel receives the fluid sample from one or more inlet ports of the microfluidic device. An outlet tube is connected to outlet port of the micro-gas exchanger. A controlled gas flow takes place in the space between the outer and inner walls and fluid flows inside the microchannel. When the fluid sample is blood, deoxygenation of the sample occurs due to gas diffusion (5% CO₂ and 95% N₂) through the inner gas-permeable wall.

It can be expected that a microfluidic device and system described herein is applicable to the study or simulation of cell heterogeneity, deformability, and adherence within subjects in larger clinically diverse populations and may provide important insights into complex disease phenotypes. For example, abnormal RBC deformability and/or adhesion to microvascular surfaces has previously been implicated in multi-system diseases, such as sickle cell disease (SCD), β-thalassemia, diabetes mellitus, hereditary spherocytosis, polycythemia vera, and malaria.

By way of example, sickled RBC occlusion or adherence to blood vessel walls has been shown to take place in post-capillary venules. To this end, this application contemplates a microfluidic testing method utilizing pathophysiologic correlates, including but not limited to, analyses of deformability and/or adhesion of RBCs, at baseline and during vaso-occlusive crises, with treatment, and in the presence of end-organ damage. The testing method described herein can be completed in less than ten minutes. In some examples, the testing method provides a highly specific analyses of the properties of RBCs, WBCs, circulating hematopoietic precursor cells and circulating endothelial cells. In one example, the testing method is performed using a miniscule blood sample (<15 μL). The testing method can provide a sophisticated and clinically relevant strategy with which patient blood samples and/or blood cells may be serially examined for cellular/membrane/adhesive properties during disease progression.

The microfluidic system can evaluate membrane and cellular abnormalities by interrogating a number of recognized abnormalities in a range of clinical phenotypes. To date, these phenotypes are discussed in various correlative blood cell studies ranging between clinical reports, testing results, interventions, and/or chart reviews.

The system described herein have advantages because existing conventional methods cannot assess longitudinal and large-scale blood cell clinical correlations with cellular, membrane, and adhesive properties. To this end, this application contemplates a method for using an the microfluidic for examining cellular properties and interactions. These cellular properties and interactions include, but are not limited to, RBC cellular and adhesive properties, WBC cellular and adhesive properties, circulating endothelial characteristics, hematopoietic precursor cell characteristics, and vascular occlusion. A simple test for blood cell deformability, occlusion, and adhesion allows exploration of its role in chronic complications in SCD, in addition to during crisis.

In some embodiments, the microfluidic system can be used in methods for analyzing, characterizing and/or predicting the deformability of cells, such as RBCs and WBCs, as well as the adherence of such cells to various capturing agents, such as such as laminin, fibronectin, E-Selectin, P-Selectin, L-selectin, intracellular adhesion molecule 1 (ICAM-1), or vascular cellular adhesion molecule 1 (VCAM-1), provided in the microchannel of the microfluidic device. In further embodiments, methods and devices are provided for diagnosing, assessing, characterizing, evaluating, and/or predicting disease based on the deformability of the cells or adherence of the cells to the capturing agents in microchannels as well as the viscosity of a fluid sample, such as blood.

Any appropriate condition or disease of a subject may be evaluated using the methods, systems, and devices described herein, typically provided that a cell may be obtained from the subject that has a material property (e.g., deformability, adherence, etc.) that is indicative of the condition or disease. The condition or disease to be detected may be, for example, a hematological disorder, such as hematological cancer, anemia, infectious mononucleosis, HIV, malaria, leishmaniasis, sickle cell disease (SCD), babesiosis, spherocytosis, monoclonal gammopathy of undetermined significance or multiple myeloma. Examples of hematological cancer include, but are not limited to, Hodgkin's disease, Non-Hodgkin's lymphoma, Burkitt's lymphoma, anaplastic large cell lymphoma, splenic marginal zone lymphoma, hepatosplenic T-cell lymphoma, angioimmunoblastic T-cell lymphoma (AILT), multiple myeloma, Waldenstrom macroglobulinemia, plasmacytoma, acute lymphocytic leukemia (ALL), chronic lymphocytic leukemia (CLL), B cell CLL, acute myelogenous leukemia (AML), chronic myelogenous leukemia (CML), T-cell prolymphocytic leukemia (T-PLL), B-cell prolymphocytic leukemia (B-PLL), chronic neutrophilic leukemia (CNL), hairy cell leukemia (HCL), T-cell large granular lymphocyte leukemia (T-LGL) and aggressive NK-cell leukemia. The foregoing diseases or conditions are not intended to be limiting. It should thus be appreciated that other appropriate diseases or conditions may be evaluated using the methods disclosed herein.

Methods are also provided for detecting and characterizing a leukocyte-mediated condition or disease. For example, methods are provided for detecting and characterizing a leukocyte-mediated condition or disease associated with the lungs of a subject being highly susceptible to injury, possibly due to activated leukocytes with altered deformability, having altered ability to circulate through the pulmonary capillary bed. Methods such as these, and others disclosed herein, can also be applied to detect and/or characterize septic shock (sepsis) that is associated with both rigid and activated neutrophils. Such neutrophils can, in some cases, occlude capillaries and damage organs where changes in neutrophil cytoskeleton are induced by molecular signals leading to decreased deformability.

In some embodiments, methods described herein can provide measurement of deformability of cell population by measuring occlusion of the cells through the microchannel. The measured occlusion of cells, such as RBCs, in the microchannels can be used to generate a red blood cell occlusive index (ROI) that is indicative of deformability of the RBCs and increased risk of vaso-occlusive crises (VOC) and/or microvascular health and function. By way of example, the ROI can be equal to the summation of the measured number of occluded RBCs in one micropillar array multiplied by size the of microcapillaries within the array divided by 4 μm. In some embodiments, the measured ROI can be compared to a control value. The subject can have an increased risk of vaso-occlusive crises (VOC) and/or decreased microvascular health and function when the ROI is greater than the control value.

A “control value” or “appropriate standard” is a standard, parameter, value or level indicative of a known outcome, status or result (e.g., a known disease or condition status). A control value or appropriate can be determined (e.g., determined in parallel with a test measurement) or can be pre-existing (e.g., a historical value, etc.). For example, a control value or appropriate standard may be the ROI of cells obtained from a subject known to have a disease, or a subject identified as being disease-free. In the former case, a lack of a difference between the measured ROI and the ROI of an appropriate standard may be indicative of a subject having a disease or condition. Whereas in the latter case, the presence of a difference between the measured ROI and the ROI of the control value or appropriate standard may be indicative of a subject having a disease or condition. While the control value or appropriate standard is described herein as being based on ROI, the control value or appropriate is not so limited and can include any mechanical property or rheological property of a cell obtained from a subject who is identified as not having the condition or disease or can be a mechanical property or rheological property of a cell obtained from a subject who is identified as having the condition or disease.

The magnitude of a difference between a parameter, level or value and an appropriate standard that is indicative of known outcome, status or result may vary. For example, a significant difference that indicates a known outcome, status or result may be detected when the level of a parameter, level or value is at least 1%, at least 5%, at least 10%, at least 25%, at least 50%, at least 100%, at least 250%, at least 500%, or at least 1000% higher, or lower, than the appropriate standard. Similarly, a significant difference may be detected when a parameter, level or value is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than the level of the appropriate standard. Significant differences may be identified by using an appropriate statistical test. Tests for statistical significance are well known in the art and are exemplified in Applied Statistics for Engineers and Scientists by Petruccelli, Chen and Nandram Reprint Ed. Prentice Hall (1999).

Further, certain methods described herein provide for measurement of adhesive properties of a cell population, in combination with or separate from measurement of the deformability or occlusion of the cell population. The combination of determining cytoadhesive properties and the deformative properties of a cell population, particularly a cell population containing a plurality of different cell types (e.g., RBCs and WBCs), may be used to generate a “Health Signature” that comprises an array of properties that can be tracked in a subject over a period of time. Such a Health Signature may facilitate effective monitoring of a subject's health over time. Such monitoring may lead to an early detection of potential acute or chronic infection, or other disease, disorder, fitness, or condition. In some cases, further, knowledge of the overall rheology of a material, along with either the deformative or cytoadhesive property of a cell, allows the determination of the other property.

In some embodiment, the deformability, occlusion, and/or adherence of cells perfused through the microchannel of the microfluidic device can be used for evaluating, assessing, monitoring, and/or predicting disease status, disease prognosis, treatment course (e.g., therapeutic selection, dosing schedules, administration routes, etc.), response to treatment and/or treatment efficacy.

In some embodiments, the microfluidic device described herein can be used to assess the health of any of the subjects described herein, used to detect or determine the stage of any of the diseases or conditions described herein and can be used for determining the number of diseased versus healthy cells.

In other embodiments, a method for detecting a condition or disease in a subject can include obtaining cells, such as a RBCs, WBCs, stem cells, or plasma cells, from the subject and perfusing a fluid containing the cells through the microfluidic channel that includes the microcapillaries and optionally various capturing agents provided in or functionalized to the microchannels.

The cells can be obtained directly or indirectly by acquiring a biological sample from a subject. For example, a biological sample may be obtained (e.g., at a point-of-care facility, e.g., a physician's office, a hospital, laboratory facility) by procuring a tissue or fluid sample (e.g., blood draw, marrow sample, spinal tap) from a subject. Alternatively, a biological sample may be obtained by receiving the biological sample (e.g., at a laboratory facility) from one or more persons who procured the sample directly from the subject. The biological sample may be, for example, a tissue (e.g., blood), cell (e.g., hematopoietic cell such as hematopoietic stem cell, leukocyte, or reticulocyte, stem cell, or plasma cell), vesicle, biomolecular aggregate or platelet from the subject.

The deformability, occlusion, and/or adherence of cells, such as RBCs and WBCs in the microchannels of the microfluidic device can then be determined and compared to a standard or control to indicate whether the subject has the condition or disease; and optionally, diagnosing the subject as having the condition or disease based on the results. The appropriate standard or control can be the number of occluded and/or adhered cells in the microchannel that were obtained from a subject who is identified as not having the condition or disease. The fluid viscosity in the microchannel can also be measured and compared to a control or standard to indicate or further characterize whether the subject has the condition or disease.

Other embodiments described herein relate to a method of assessing microvascular health and function of a subject in need thereof. The method can include perfusing a fluid sample including RBCs from the subject through the at least one microchannel of a microfluidic device described herein. The number of occluded RBCs in the at least one microchannel can then be measured. A RBC occlusive index (ROI) can be generated from the measured number of occluded RBCs. The ROI can be indicative of increased risk of vaso-occlusive crises (VOC) and/or microvascular health and function

In some embodiments, the ROI can be compared to a control value. The subject can have an increased risk of vaso-occlusive crises (VOC) and/or decreased microvascular health and function when the ROI is greater than the control value.

In some embodiments, the fluid sample can be perfused under at least one normoxic or hypoxic conditions and the number of RBCs can be measured using an imaging system.

Still other embodiments relate to a method of assessing the pathology of RBCs. The method can include perfusing a fluid sample including the RBCs through the at least one microchannel of a microfluidic device described herein. The number of occluded RBCs can be measured in the at least one microchannel. A red blood cell occlusive index (ROI) can be generated from the measured number of occluded RBCs. The ROI can be indicative of the number of pathologically impaired RBCs.

In some embodiments, the RBCs are from a subject at risk of a vaso-occlusive crises and/or decreased microvascular health and function. The subject has an increased risk of vaso-occlusive crises (VOC) and/or decreased microvascular health and function when the ROI is greater than the control value. In some embodiments, subject can have or be at an increased risk of malaria or sickle cell disease. The RBCs can be from stored blood and/or blood to be transfused and the ROI can be used to determine the fitness or storage lesions of the stored RBCs and/or RBCs to be transfused.

Still other embodiments relate to a method of measuring efficacy of therapeutic agent in modulating blood cell adhesion and/or deformability. The method can include perfusing a fluid sample including blood cells through the at least one microchannel of a microfluidic device described herein. The number of occluded blood cells in the at least one microchannel can then be measured. The therapeutic agent can be added to at least one of the fluid sample prior to perfusion through the at least one microchannel or before and/or during perfusion of the fluid sample through the at least one microchannel.

In some embodiments, the efficacy of the therapeutic agent based can be determined based on the measured number of occluded blood cells. A decrease in the measured number of occluded blood cells compared to a control is indicative of the therapeutic agent having an increased efficacy in decreasing blood cell adhesion and/or increasing blood cell deformability.

Still other embodiments relate to a method of separating plasma from whole blood with any or minimal RBC lysis. As illustrated in FIG. 5, the method can include sedimenting RBCs in a whole blood sample without lysing the RBCs. In some embodiments, the sedimentation of the RBCs can be accelerated by adding fibrinogen to a whole blood sample. The sedimented red blood cells can then be separated from plasma in the whole blood sample. A red blood cell stiffener, such as a diamide, can then be added to the sample and the plasma can be perfused through at least one microchannel of the microfluidic device to occlude any remaining red blood cells so that the plasma is free of or substantially free of RBCs.

In some embodiments, the microfluidic device can include a micropillar array at the second end that defines a plurality of microcapillaries that each have cross sectional area effective to occlude the passage of red blood cells but not plasma.

### Example 1

In this example, we designed a novel microfluidic platform, the OcclusionChip, featuring embedded micropillar arrays forming uniform microcapillaries within each array but overall finer microcapillaries along the flow direction from 20 μm down to 4 μm, coupled with two 60-μm side passages, which mimics many features of the human capillary bed (FIGS. 1A & B) and allows the assessment of large quantity of RBCs in a high throughput manner.

We designed the OcclusionChip such that RBCs with significantly impaired deformability are retained by coarser upstream micropillar arrays with coarser microcapillaries, while those with moderately impaired deformability are retained by finer downstream micropillar arrays. Compared to many existing microfluidic approaches that quantify RBC deformability based on velocity or other parameters, our device quantifies mechanical retention, which may better reflect pathophysiological processes in which poorly deformable RBCs obstruct capillaries, leading to microvasculature dysfunction. Here, we present the design, fabrication, and validation of the OcclusionChip to assess and quantify microvascular occlusion mediated by RBCs, and we define a new term, RBC Occlusion Index, which may serve as an in vitro test of microvascular health and function.

### Materials and Methods

### Occlusionchip Fabrication and Assembly

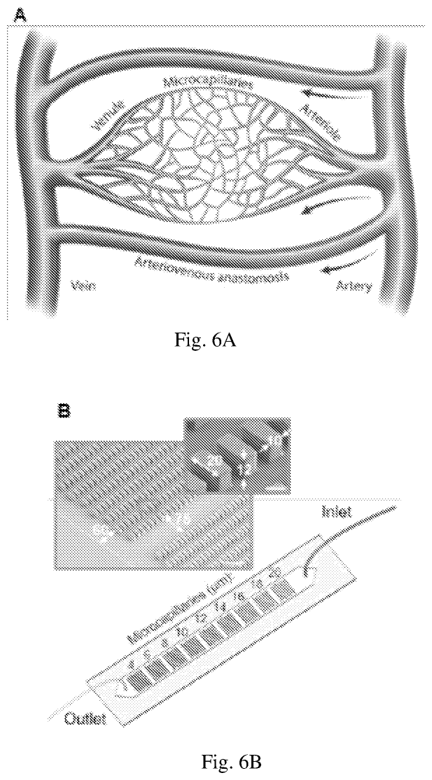

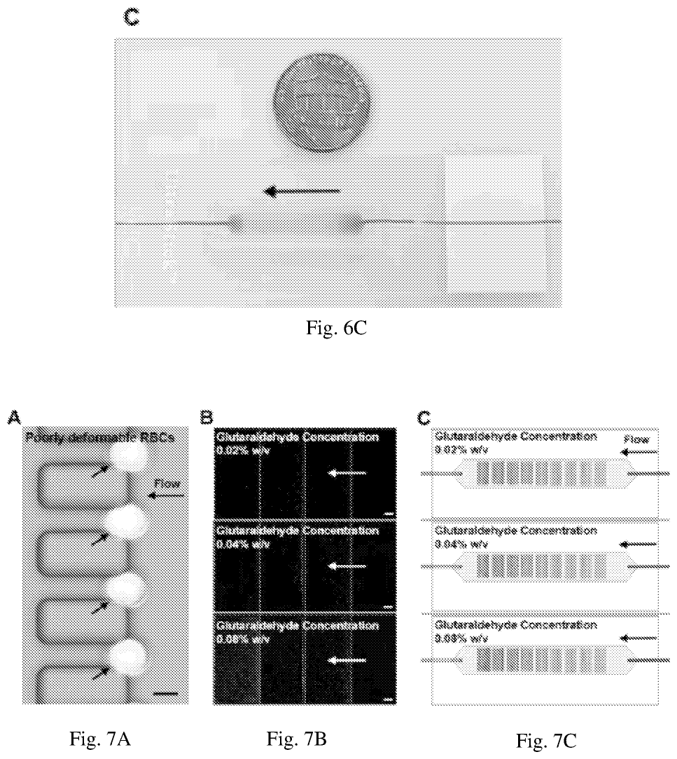

The OcclusionChip was fabricated based on standard lithography techniques (FIG. 6). A negative template on a 3-inch silicon wafer (University Wafers, Boston, Mass.) was initially fabricated through photolithography. Briefly, a layer of negative photoresist SU8-2010 (Thermo Fisher Scientific, Waltham, Mass.) was spin-coated on the wafer at a thickness of 12 μm. After being soft-baked at 95° C. for 4 min, the wafer was exposed to UV light with alignment to selectively cure the photoresist. Following the post-exposure bake, in which the wafer was baked in the same condition as soft-bake, the wafer was developed in a photoresist solvent propylene glycol monomethyl ether acetate (PGMEA, Sigma Aldrich, St. Louis, Mo.), and hard-baked at 110° C. overnight. A 2-hour surface passivation using trichloro (1H, 1H, 2H, 2H-perfluorooctyl) saline (Sigma Aldrich, St. Louis, Mo.) was performed under vacuum to facilitate the separation of the molded polymer from the master wafer. Next, a polydimethylsiloxane (PDMS, Thermo Fisher Scientific, Waltham, Mass.) pre-polymer was mixed with the curing agent at a ratio of 10:1 (v/v) and degassed in a desiccator to remove any air bubbles. The mixture was poured over the master wafer and cured at 80° C. overnight. Two 0.5 mm-diameter holes were punched as the inlet and the outlet after the PDMS block was separated from the master wafer. Excessive saline was removed by sonicating with isopropanol for 5 min. Tubing was assembled after the PDMS block was bonded to a microscope glass slide (Microscopy Sciences) through surface modification under oxygen-plasma treatment. The fabricated microchannel was rinsed in 100% ethanol and phosphate-buffered saline (PBS), and incubated with 2% bovine serum albumin (BSA, ProSpec-Tany TechnoGene Ltd, East Brunswick, N.J.). Prior to introducing blood samples, the microchannel was rinsed with PBS to remove excessive BSA.

### Blood Sample Collection and Preparation

All blood samples were collected following the procedures in accordance with an Institutional Review Board (IRB) approved protocol. Informed consent was obtained from all subjects. Blood samples from healthy donors (HbAA) and SCD subjects (homozygous HbSS and heterozygous HbSC) were anticoagulated with ethylenediaminetetraacetic acid (EDTA) and stored at 4° C. Healthy RBCs were treated with glutaraldehyde, Diamide, and mercuric ion according to protocols detailed in Supplementary Materials. Plasmodium falciparum-infected RBCs (pf-iRBCs) were cultured at 4% hematocrit under standard conditions in RPMI 1640 according to published protocols. Parasitized RBCs were labeled with Hoechst 33342 for 30 minutes in room temperature. Stored blood samples were obtained from Hemanext (Avon, Mass.). Briefly, packed RBCs were prepared from whole blood anticoagulated with citrate-phosphate-double dextrose (CP2D) anticoagulant after centrifugation and removal of the plasma. The RBCs were re-suspended in AS3 red cell storage solution and stored in conventional storage bag for 42 days at 4° C. Samples were removed every 7 days for OcclusionChip testing. Lactate production and glucose consumption were analyzed to determine the metabolic activities of the stored RBCs. Hemolysis levels were well below FDA guidelines of 1%, maximum allowable limit for red cell product for transfusion. Blood samples from patients on hemodialysis were obtained from the clinics at University Hospitals, with IRB approval.

### Microfluidic Assessment of Blood Samples

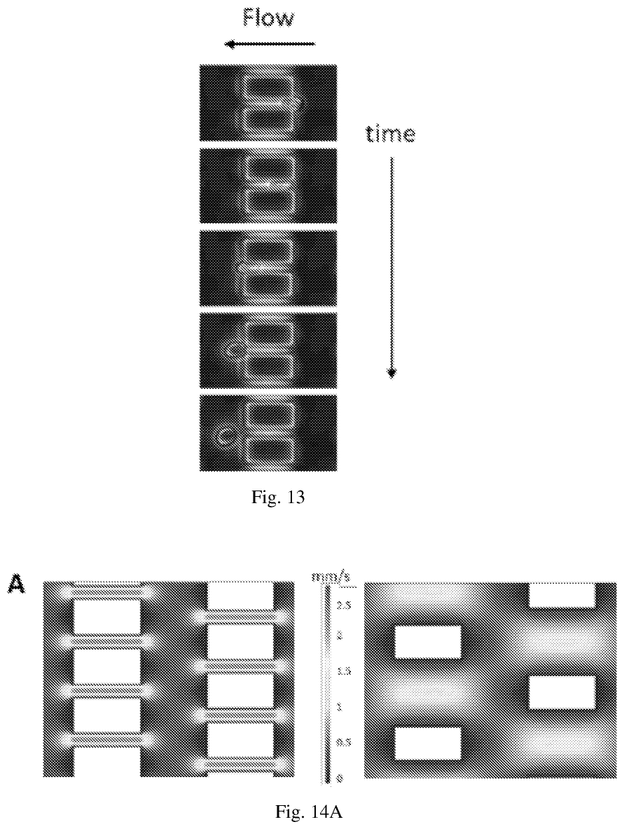

Whole blood samples were centrifuged at 500×g for 5 min at 4° C., plasma was removed, and the RBCs were washed twice in PBS, and re-suspended at a 20% hematocrit value. The OcclusionChip was placed on an Olympus IX83 inverted motorized microscope stage for high-resolution imaging. The RBC samples were perfused through the microchannel under constant pressure for 20 min (FIGS. 12A & B). Non-retained RBCs were rinsed away by injecting PBS into the microchannel under the same pressure. FIG. 13 depicts an RBC traversing a 4-μm microcapillary and shows RBCs passing through the OcclusionChip micropillar arrays. Negligible contamination by platelets and white blood cells was observed by visual inspection. Phase contrast, bright field, and fluorescent images were recorded at 20× using the Olympus Cell Sense live imaging software (excitation/emission wavelength, 488/505-580 for Green Fluorescent Protein, GFP). Post processing of recorded images and cell counting were performed using Adobe Photoshop (San Jose, Calif.). Oxygen tension was controlled in hypoxia experiments using a custom designed gas exchange setup as described in Supplementary Materials. Scanning electron microscopy was performed on the micropillars and RBCs.

### Statistical Analysis

The data are reported as mean±standard error of the mean (SEM) in this study. A test of normality was performed. One-way ANOVA was used for normally distributed data with Tukey's post hoc analysis for more than two groups. Non-parametric Mann-Whitney U-test or Kruskal-Wallis H tests with Dunn's post hoc analysis for comparing more than two groups for non-normally distributed data. Statistical significance was defined with p-value less than 0.05 (p<0.05). Statistical analyses were performed using Minitab 19 (Minitab, Inc., State College, Pa.) and Matlab (Mathworks, Mass., USA).

### Results

### Occlusionchip Design and Testing

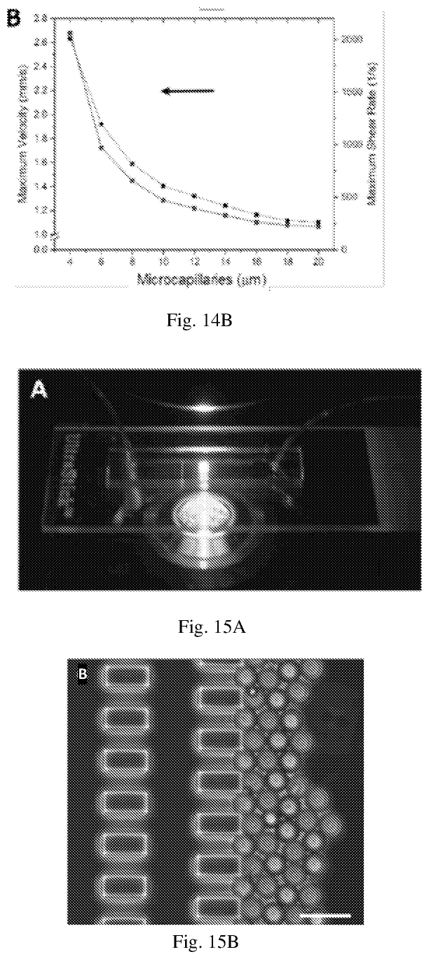

The fabricated and assembled OcclusionChip is shown in FIG. 6C. Inspection and characterization of the fabricated PDMS block with micropillar features were achieved by scanning electron microscopy, from which the geometry and dimension of the fabricated PDMS block with microcapillary dimensions were confirmed (FIG. 6B insets). Briefly, the fabricated PDMS micropillars are of 12-μm height, 20-μm length and 10-μm width. Each micropillar array is 2-mm long and the distance between two successive micropillar arrays is 75 μm. Two 60-μm wide paths on both sides were designed to mimic anastomosis of human capillary bed to prevent complete microchannel blockage (FIG. 6A). The flow environment was characterized via a 2-D numerical simulation performed by a commercially available FEA software package COMSOL 4.3 (Burlington, Mass.) with the assumption of Newtonian behavior. The boundary conditions at the inlet and outlet were set to pressure inlet and pressure outlet respectively, where the RBC suspension was modeled with a dynamic viscosity and density of 0.001 Pa·s and 993 kg/m³. The possible effects of fluid-structure interactions on the flow were neglected in the simulation by treating PDMS structures as rigid bodies. Typical velocity distributions are shown at the micropillar array (FIG. 14A), as is estimated maximum velocity and shear rate values across the microchannel (FIG. 14B).

Validation of OcclusionChip Micropillar Arrays with Rigid Fluorescent Microbeads

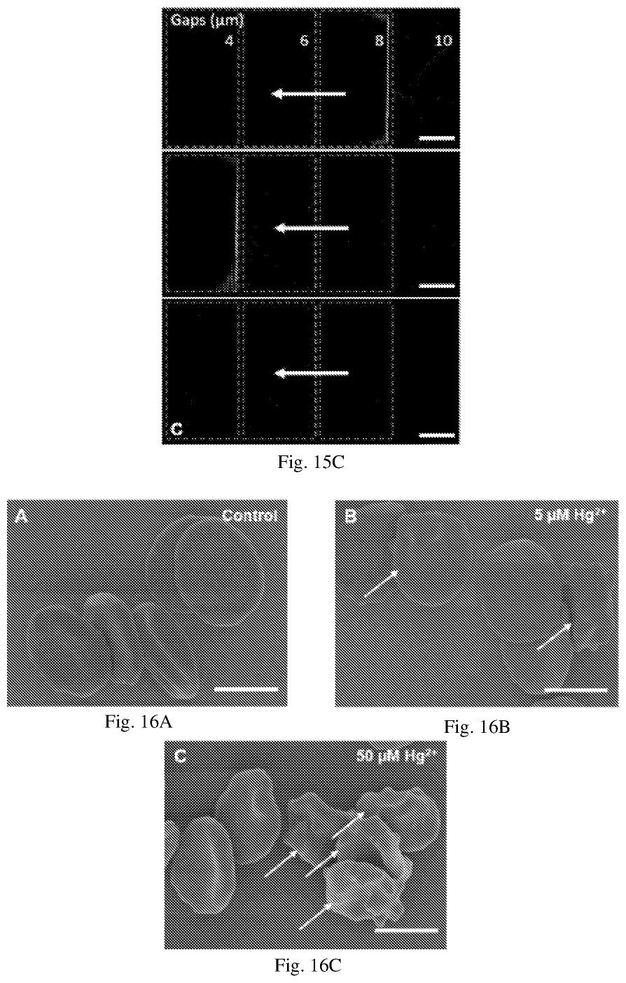

The functional validation of the OcclusionChip was carried out using rigid fluorescent microbeads (Polysciences Inc., Warrington, Pa.) at 4, 6, or 10-μm diameter. Solutions containing fluorescent microbeads were prepared by mixing the microbead stock solution with BlockAid blocking solution (Thermo Fisher) at a ratio of 1:200 v/v. A visually clear line was observed by the blockage of the 10-μm microbeads at the beginning of the micropillar array with 8-μm gaps (FIG. 15A). The same field of view of all the three tests using the three different diameters of microbeads are shown, in which the microbeads were retained within gaps smaller than their outer diameter (FIGS. 15B & C). This result further confirmed that the micropillar arrays served as a retention mechanism, which is essential to RBC deformability assessment.

Mechanical Retention of Chemically Treated RBCs with Glutaraldehyde, Diamide, or Mercuric Ions, and RBC Occlusion Index as a Biomarker to Assess RBC Deformability

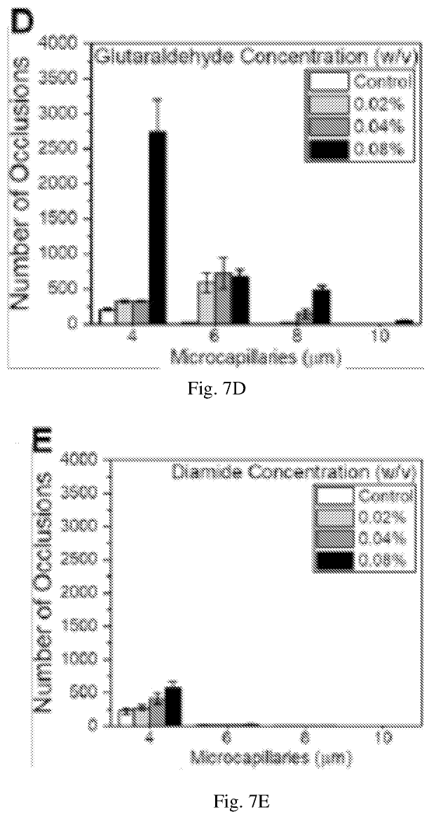

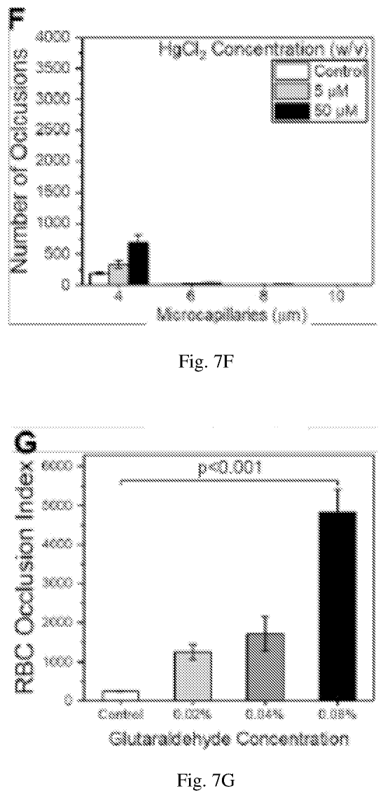

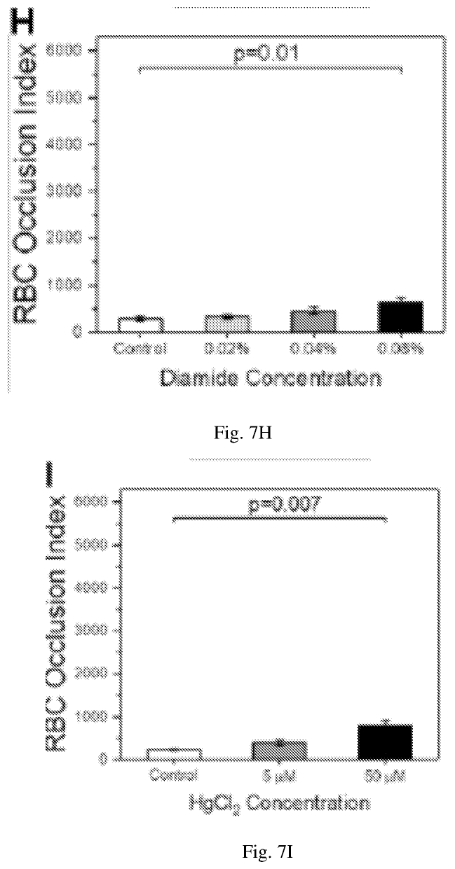

Glutaraldehyde, a non-specific protein cross-linker, and diamide, a thiol group oxidant inducing disulfide cross-linking of band 3, are used to stiffen RBCs in order to mimic impaired RBC deformability, seen in pathological conditions. Precisely controlled exposure of RBC to graded concentrations of glutaraldehyde or diamide was utilized to verify OcclusionChip functionality. RBCs from healthy donors exposed to graded concentrations, 0.00% (as control), 0.02%, 0.04%, and 0.08% (w/v), of glutaraldehyde were mixed with untreated normal RBCs to achieve 1% treated cells and were suspended in PBS for microfluidic assessment. RBCs exposed to same graded concentrations of diamide were directly suspended in PBS for microfluidic assessment. RBCs with impaired deformability were mostly retained at the 4-μm microcapillaries (FIG. 7A), with a smaller number retained at the 6, 8, and 10-μm microcapillaries, later in the assay (not shown). No appreciable occlusion was observed elsewhere. The retained RBCs were fluorescently labeled with anti-glycophorin A (CD235a, Abcam, Cambridge, Mass.) antibodies. Results obtained with RBCs from the same blood sample that was exposed to graded concentrations of glutaraldehyde are shown (FIG. 7B, control not shown). Of note, the distribution of retained RBCs within the channel revealed a unique aspect of the microfluidic device: it generated distinct patterns in response to the heterogeneous RBC deformability that was induced by various concentrations of glutaraldehyde (FIG. 7C). At the end of the test, numbers of occlusions induced by poorly-deformable RBCs within each micropillar array were quantified (FIGS. 7D&E). The number of occlusions induced by poorly deformable RBCs increased as the concentration of glutaraldehyde or diamide increased. The maximum difference in total occlusion level was observed with glutaraldehyde treatment at a concentration of 0.08% (w/v), where the rate of increase was more than 16-fold compared to the control. To assess the overall RBC deformability of specific samples, we assigned different weight to the occlusion events quantitated within different micropillar arrays, and defined a new parameter ‘RBC Occlusion Index’ (ROI) using the following equation,

$$RBC Occlusion index = Σ the number of occlusions in one array \times \frac{the size of microcapoilaries within the array}{4 μ m} \qquad ( 1 )$$

Our results showed that the ROI generated by RBCs exposed to glutaraldehyde or diamide increased in a concentration dependent manner (glutaraldehyde: FIG. 7G, p<0.001, one-way ANOVA, mean±SEM=245.7±20.7 control, 1245.3±197.7 0.02%, 1714.1±444.9 0.04%, 4826.6±582.3 0.08% and diamide: FIG. 7H, p=0.01, one-way ANOVA, mean±SEM=292.6±46.3 control, 329.9±37.2 0.02%, 459.8±71.6 0.04%, and 645.1±87.6 0.08%, which indicates that the defined ROI could reflect the deformability of the entire RBC population.

Mercury is a toxic heavy metal that binds to RBC membranes with high affinity and results in RBC hemolysis. Mercuric ions induce membrane conformational changes and impair RBC deformability. In this study, we interrogated the effect of short-term exposure of mercuric ion on RBC deformability, at both a low (5 μM Hg²⁺) and high level (50 μM Hg²⁺), as the range of the mercury level in vivo has been reported at 16,000 μg/L (58 μM) to 11 μg/L (0.055 μM) in blood. Hg²⁺-exposed RBC samples were perfused through the OcclusionChip, and enhanced retention of poorly deformable RBCs was observed at the 4-μm microcapillaries for all Hg²⁺-treated samples (FIG. 7H), suggesting moderate impairment in RBC deformability following short-time mercuric ion exposure. The ROI generated by Hg²⁺-exposed RBCs increased in a mercuric ion concentration dependent manner (FIG. 7I, p=0.007, Kruskal-Wallis, median±SEM=211±24.8 control, 341±60.1 5 μM, and 712±118.6 50 μM mercury. Scanning electron microscopy result confirmed that mercury induced morphologic changes in the RBC, including the loss of the characteristic biconcave shape and the formation of spiked cell membrane (FIG. 11). Detailed sample processing for scanning electron microscopy is summarized in Supplementary Information. Taken together, our results demonstrate that short-term mercuric ion exposure induces RBC morphology change and moderately reduces RBC deformability.

### RBC Occlusion Index as a Biomarker to Assess Pathologically Impaired RBC Deformability in Sickle Cell Disease

We next demonstrate the pathophysiological relevance of the microfluidic device using RBCs from subjects with SCD. In SCD, deoxy-sickle hemoglobin may undergo intracellular hemoglobin polymerization, which results in RBC morphology change and impaired deformability. We assessed RBCs from 16 individuals with SCD with homozygous HbSS, and RBCs from 5 healthy donors (HbAA). A representative distribution of retained HbSS RBCs within the OcclusionChip is shown in FIG. 8A. The morphology of retained HbSS RBCs at two selected micropillar arrays are also shown in FIG. 8A insets. The close-up view shows that, the occlusions of 4-μm microcapillaries were induced by a morphologically heterogeneous RBC population from subjects with SCD, including disc-shaped (indicated in orange zone), mildly sickled (indicated in rose zone), and highly sickled (indicated in red zone) cell morphologies within the same field of view. On the other hand, we did not observe morphologically heterogeneous cell populations within normal RBCs (not shown). Notably, HbSS RBCs appeared to stagnate around the front surface of the micropillars due to the local flow environment (the dead zone showed in FIG. 14A). The numbers of occlusions induced by HbSS RBCs across the device were quantified (FIG. 8B), from which the average ROI of HbSS RBCs is significantly higher compared to HbAA RBCs (FIG. 8C, mean±SEM=1832.4±281.6 vs. 115.1±25.4, respectively, p=0.001, Mann-Whitney), suggesting that HbSS RBCs are inherently less deformable than HbAA RBCs.

### Hypoxia Enhanced Morphological Changes in RBCs in Sickle Cell Disease, and ROI as a Biomarker to Assess Abnormal RBC Deformability Mediated by Hypoxia

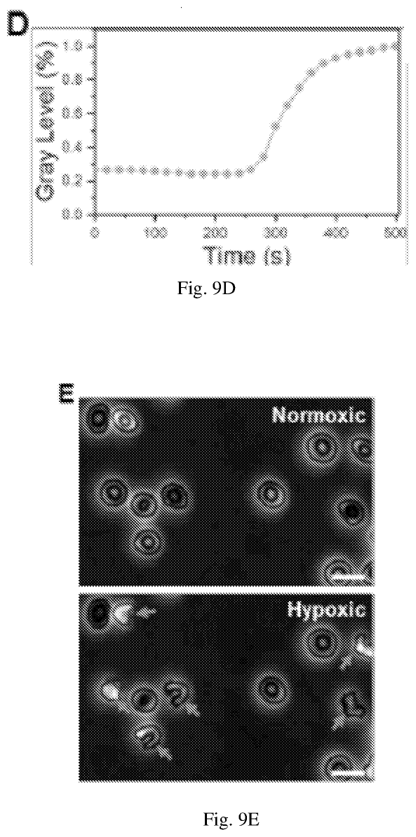

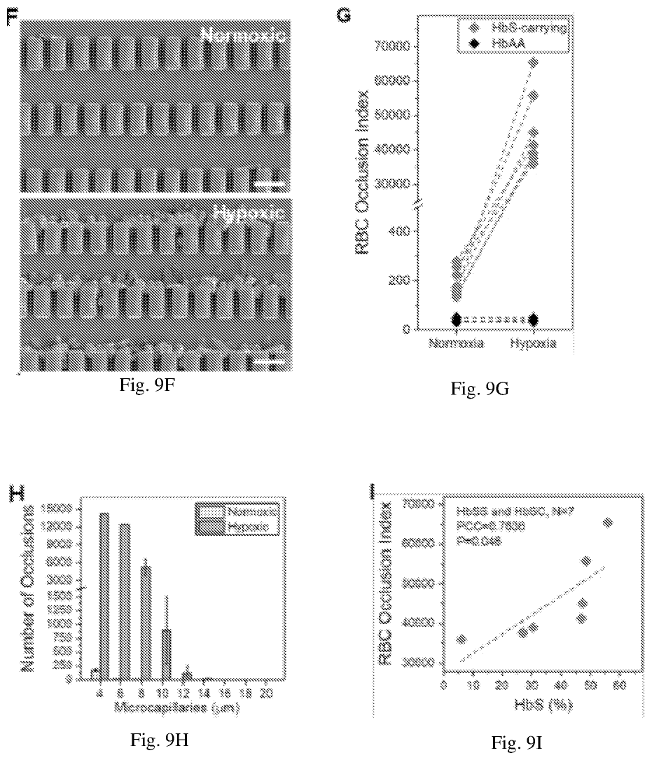

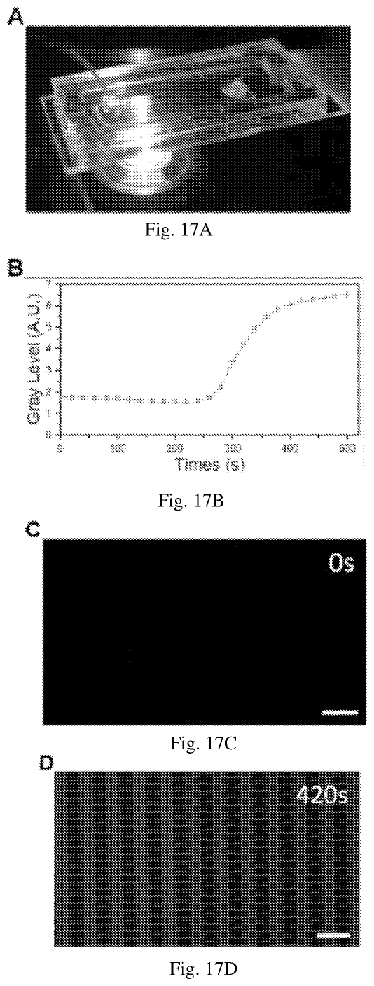

In order to create a hypoxic condition within the microchannel to study the behavior of RBCs in post-capillary networks in SCD, a deoxygenation system was coupled with the OcclusionChip (FIG. 9A). We utilized a custom gas exchanger to deoxygenate the blood flow developed in one of our previous studies (FIG. 9B), which desaturates hemoglobin in the sample to an approximate SpO₂ of 83%. We designed custom gas exchange tubing and gas exchange microchannel to stabilize the oxygen level within the microchannel as the gas permeability of PDMS was recognized (FIG. 9C). The detailed fabrication process is presented in Supplementary information. Verification of oxygen diffusion was performed by introducing an oxygen-sensitive luminescence probe tris(4,7-diphenyl-1,10-phenanthroline)ruthenium(II) dichloride complex (Santa Cruz Biotechnology, Dallas, Tex.) (1 mg/ml in PBS) into the microchannel (FIG. 17). Molecular oxygen can quench the luminescence of this probe; thus, the dissolved oxygen level can be measured by the luminescent intensity. In our experiment, a controlled gas flow (95% N₂ plus 5% CO₂) was allowed at 0 s and the luminescence was monitored under illumination at 488 nm. Gas exchange was completed at approximately 420 s as the luminescent intensity saturated (FIG. 9D). RBCs from 7 subjects with SCD (4 homozygous HbSS and 3 compound heterozygous HbSC individuals, all termed as HbS-carrying) and 3 healthy donors were tested under both ambient and hypoxic conditions. Although it has been reported that the sickling of HbS-carrying RBCs depends on the blood oxygen level, here we observed heterogeneous levels of cell sickling and changes in morphology under identical degrees of hypoxia (FIG. 9E). As expected, HbA-containing RBCs showed no change in morphology under hypoxic conditions (not shown).

We tested the effect of hypoxia on the deformability change of HbS-carrying RBCs and HbAA RBCs. The sample perfusion duration was decreased to 2 minutes in order to prevent full channel blockage. Our scanning electron microscopy results showed that when oxygen levels were reduced, HbS-carrying RBCs undergoing sickling were unable to pass through the microcapillaries, thereby inducing enhanced microchannel occlusion and resistance to blood flow (FIG. 9F). Detailed sample processing for scanning electron microscopy is summarized in Supplementary Information. The ROI was significantly increased in hypoxic sickle RBCs compared to sickle RBCs examined under ambient conditions; however, the ROI generated by HbAA RBCs did not change under hypoxia (FIG. 9G, HbS RBCs: 195.1±21.8 vs. 45,750.9±4,116.5 under hypoxia, and HbAA: 38.7±5.7 vs. 40.3±4.9 under hypoxia). Profiles of occlusions induced by HbAA RBCs and HbS-carrying RBCs in hypoxic conditions are shown, from which full blockages of the 4-μm and 6-μm micropillar arrays were observed for all HbS-carrying RBC samples (FIG. 9H). Furthermore, a strong correlation between the ROI and HbS levels of the subjects was observed (FIG. 9I, PCC=0.76, p<0.05, N=7). Of note, we observed recovery of sickled HbS-carrying RBCs as well as clearance of retained HbS-carrying RBCs within the microchannel by stopping the controlled gas flow, which demonstrates that the intracellular HbS polymerization and RBC sickling due to hypoxia is reversible, and our device is capable of detecting this effect.

RBC Occlusion Index as a Biomarker to Assess Impaired RBC Deformability Induced by Plasmodium falciparum Infection, Blood Storage Lesion, or Renal Failure

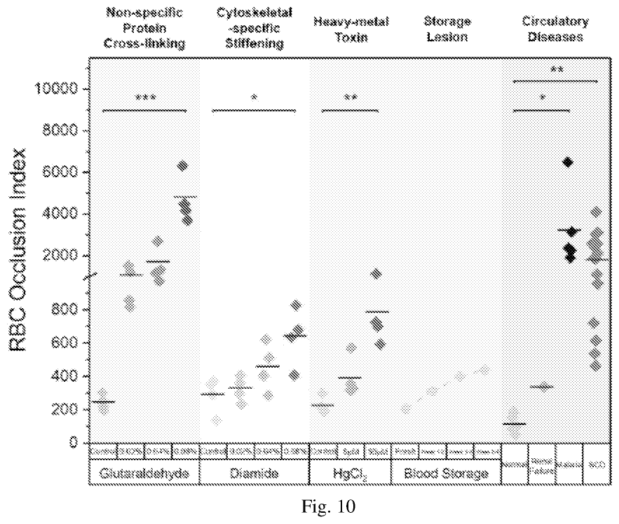

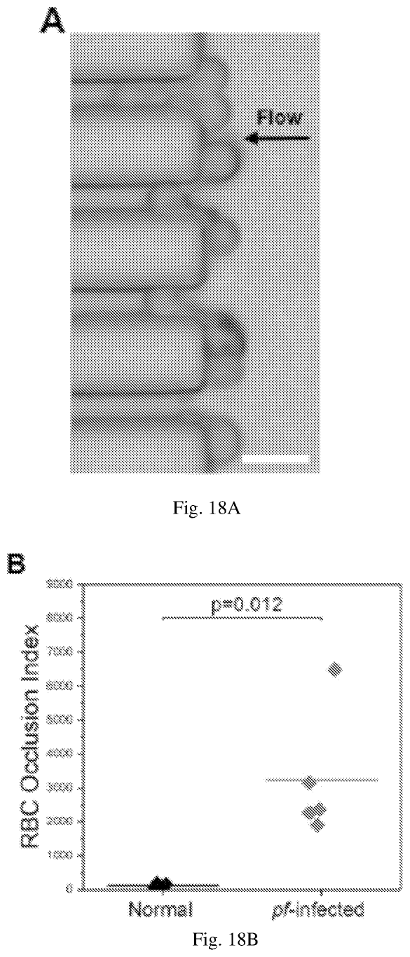

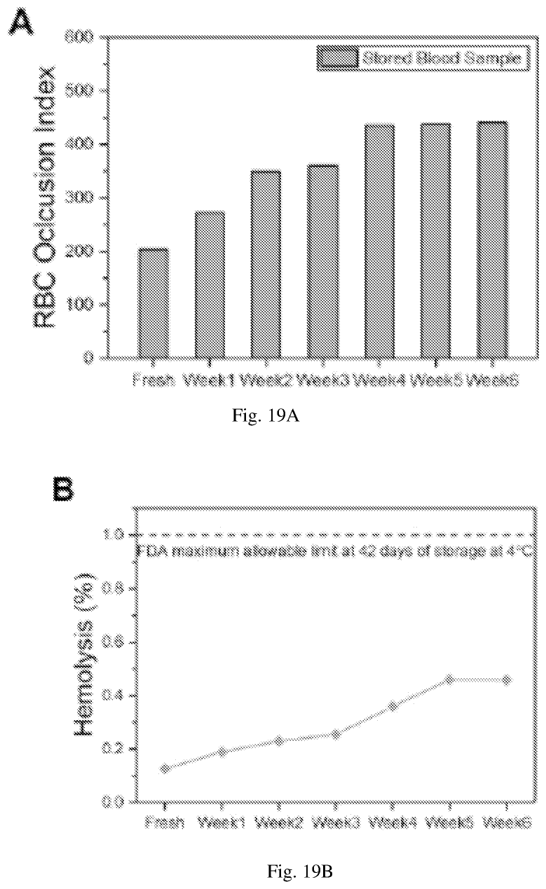

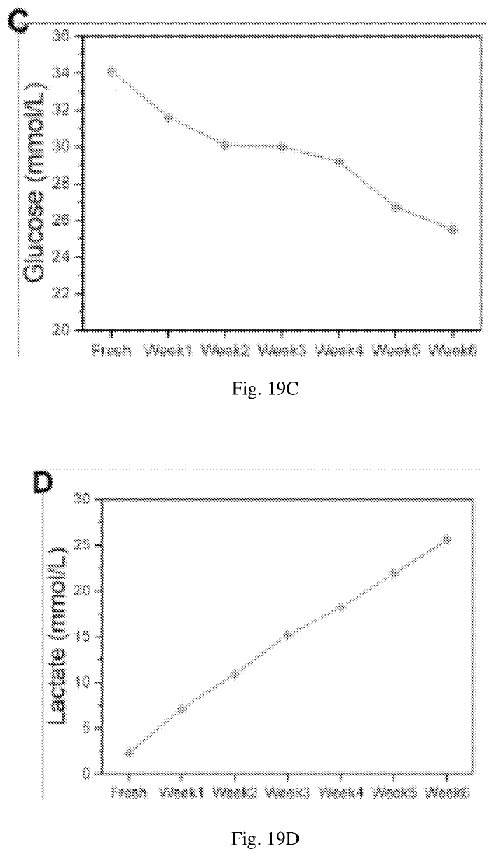

To further demonstrate the physiological relevance of our OcclusionChip, we tested RBCs that had been stored, exposed to Plasmodium falciparum infection, or came from a patient with end stage renal failure. Typical retained Hoechst-labeled pf-iRBCs within the microchannel are shown in FIG. 18. We observed that the pf-iRBCs were preferentially retained by various sizes of microcapillaries, which generated a significantly higher level of ROI, compared to normal, non-infected RBCs (FIG. 5, N=5, malaria: mean±SEM=3,232.7±838.6, and normal: mean±SEM=115.1±25.4, p=0.012, Mann-Whitney). In addition, the ROI of one blood sample stored for up to 42 days were monitored for 6 weeks, from which we observed elevated ROI as the storage progressed, indicating the progressively decreased deformability of the stored RBCs (FIG. 10, mean=212.3 vs. 311.5 vs. 397.8 vs. 439.8 for fresh, week 1-2, week 3-4, and week 5-6, respectively). The progressively reduced deformability of stored RBCs is also reflected by the hemolysis level, glucose consumption, and lactate production (FIG. 19). Furthermore, RBCs from hemodialyzed samples were assessed, from which we observed a small change of ROI due to the retention of poorly deformable RBCs primarily within the 4-μm micropillar array, suggesting a moderate reduction of RBC deformability in subjects with end-stage renal disease. (FIG. 10, N=2, mean±SEM=336.3±2.3).

In this example, we described a novel microfluidic platform, the OcclusionChip that mimics features of the human capillary bed, allowing for the real-time visualization and quantitative assessment of RBC deformability and associated microvascular occlusion under physiological flow. We focused on a realizable design that assesses RBC deformability at a single-cell level in a high-throughput manner. The primary advantage of our device over other RBC deformability measurement technologies is that RBCs perfused through the OcclusionChip experience a wide spectrum of deformations when crossing constrictions with different sizes, which recapitulates a more physiologically relevant microenvironment. Additionally, most other RBC deformability measurement technologies fail to examine large numbers of heterogeneous RBCs, and so are limited. The OcclusionChip design inherently eliminates this limitation since the embedded micropillar arrays recapitulate large numbers of microcapillaries with various dimensions, enabling the simultaneous deformability analysis of bulk RBCs at a single-cell level. The visual quantification of occlusions induced by poorly deformable RBCs that are retained by the microcapillaries within each micropillar array, and the resulting ROI make the assessment of overall RBC deformability and associated microvascular occlusion possible.

To demonstrate the physiological relevance of the OcclusionChip, we utilized precisely controlled exposure of normal RBCs to glutaraldehyde or diamide, which are widely used to verify new RBC deformability measurement techniques. We further interrogated abnormal RBC deformability that is induced by exposure to mercuric ions or in SCD. We found that short-term mercuric ion exposure altered RBC deformability in a concentration dependent manner (FIG. 10), which is in accordance with early in vitro studies on the hemolytic effect of inorganic mercuric ion on RBCs compromising membrane fragility and cell deformability. SCD is one of the most common hemoglobin disorders worldwide, and is associated with impaired RBC deformability. Here, RBCs from subjects with SCD induced significantly higher, albeit heterogeneous, microcapillary occlusions compared with normal RBCs (FIG. 10). Therefore, monitoring RBC deformability change may be of significant relevance in this disease.

The reduced deformability of deoxygenated RBCs induced by intermittent hypoxia in SCD has been studied since the 1970s. It is well established that in SCD, RBCs undergo extensive morphology alterations when exposed to progressive oxygen tension, which is accompanied by reduced deformability. The OcclusionChip integrated with the gas exchanger enables the visual analysis of RBC morphological sickling and occlusions of microcapillaries in the absence of other type of blood cells (e.g., WBCs or platelets). Therefore, we were able to analyze the independent contribution of RBCs to vaso-occlusions under hypoxia. By demonstrating that interactions between deoxygenated HbS-carrying RBCs and capillary geometries are sufficient to induce severe microvascular occlusions in the absence of multicellular adhesion events, our system provided unique insight into hypoxia-mediated SCD vasculopathy, that other in vitro systems are not able to observe. Moreover, the strong correlation between the ROI generated by deoxygenated HbS-carrying RBCs and the HbS levels of SCD subjects (FIG. 9I) also suggests that the OcclusionChip could be adapted as a personalized disease-monitoring tool, and/or utilized in phenotypic drug discovery and therapeutic strategy assessment in the circumstances involving hypoxia-mediated vaso-occlusive events.

Envisioning a universal RBC deformability assessment tool for all circulatory diseases, we further used the established ROI as a biomarker to assess the abnormal deformability of in vitro malaria infected RBCs, RBCs from patients with end-stage renal disease, and stored RBCs. Early ex vivo studies have revealed the impaired deformability of malaria infected RBCs, which is stage dependent, decreasing as intra-erythrocytic parasites mature. In full agreement with this, we observed enhanced occlusions within multiple micropillar arrays induced by malaria infected RBCs, with a markedly increased ROI compared to normal RBCs (FIG. 10, FIG. 18B). RBCs from people on hemodialysis displayed moderately impaired deformability, (with a modestly elevated ROI), which is in agreement with a previous observation that RBCs from patients with end stage renal failure required more time to pass through membrane filters compared to normal RBCs.

Finally, recent studies have demonstrated that RBCs become less deformable and harder to elongate after long-time storage. Our results showed that during the 6-week storage period, the ROI generated by the stored RBCs increased modestly with time (FIG. 5). This suggests that RBCs become stiffer and less deformable when stored, which is in accordance with the previous studies. While currently no routine tests exist in the blood bank to examine the deformability of stored RBCs, the OcclusionChip assay developed here may contribute to the detection of storage lesions and improve the outcomes of transfusion therapy.

### Example 2

This example describes a microfluidic device that assesses red blood cell (RBC) adhesion endothelial associated to adhesion molecules (such as laminin (LN), fibronectin (FN), selectins (P-, E-, or L-), intracellular adhesion molecule-1 (ICAM-1), and vascular cell adhesion molecule-1 (VCAM-1)) and RBC mediated microvascular occlusion in an integrated manner. The RBC adhesion is measured through microchannel surface functionalization technique, and the microvascular occlusion is measured through mechanical retention mechanism enabled by the microchannel geometries.

RBC adhesion and RBC deformability (defined as the ability to squeeze through narrow capillaries) are two critical factors modulating RBC ability to navigate across the microvasculature. Abnormalities in both of these two factors have been associated with vaso-occlusive events in sickle cell disease (SCD). These two factors are typically assessed using separate systems. Here, we simultaneously assessed RBC adhesion on the inflamed vascular wall and microvascular occlusion using clinical blood samples in a microfluidic device.

The microfluidic device described herein integrated with a series of micropillar arrays forming microcapillaries and surface functionalization with endothelial associated adhesion molecules, which is for concurrent assessment of RBC adhesion to laminin and RBC mediated microvascular occlusion.

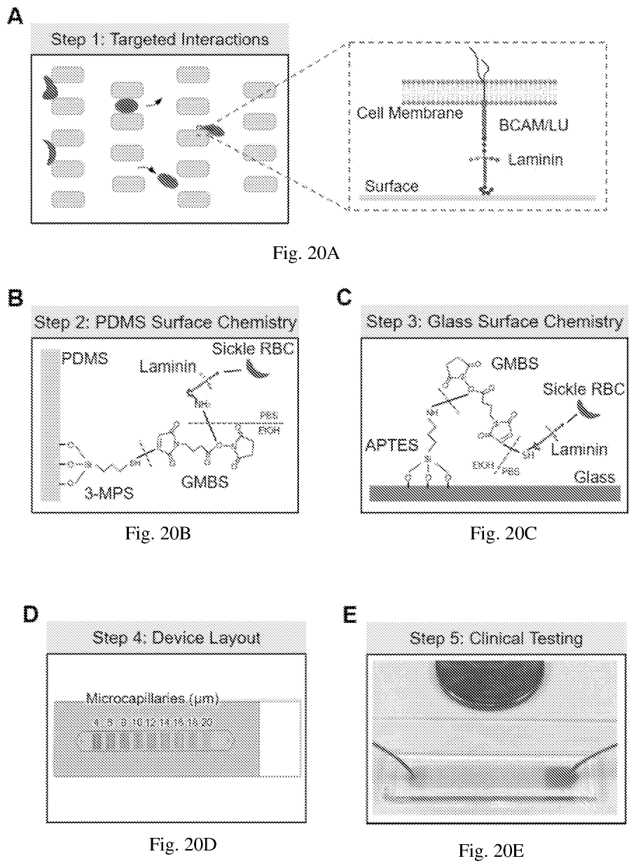

The device includes a microchannel with micropillar arrays mimicking the capillary network (FIG. 20). The microchannel surface is functionalized with LN mimicking vascular damage in SCD. The microchannel is coupled to a digital pump that provides a constant pressure value of 35 mBar to drive the RBC flow. RBC adhesion and microvascular occlusion are simultaneously assessed using the proposed device.

### Fabrication

The microfluidic device is fabricated through standard photolithography and soft lithography techniques. A negative template on a 3-inch silicon wafer is initially fabricated. Briefly, a layer of negative photoresist SU8-2010 is spin-coated on the wafer at a thickness of 12 μm. After being soft-baked at 95° C. for 4 min, the wafer is exposed to UV light with alignment to selectively cure the photoresist. Following the post-exposure bake, in which the wafer will be baked in the same condition as soft-bake, the wafer is developed in a photoresist solvent propylene glycol monomethyl ether acetate (PGMEA), and hard-baked at 110° C. overnight. A 2-hour surface passivation using trichloro (1H, 1H, 2H, 2H-perfluorooctyl) saline is performed under vacuum to facilitate the separation of the molded polymer from the master wafer. Next, polydimethylsiloxane (PDMS) pre-polymer is mixed with the curing agent at a ratio of 10:1 (v/v) and degassed in a desiccator to remove any air bubbles. The mixture is poured over the master wafer and cured at 80° C. overnight. Two 0.5 mm-diameter holes are punched as the inlet and the outlet after the PDMS block is separated from the master wafer. Excessive saline is removed by sonicating with isopropanol for 5 min. Tubing is assembled after the PDMS block is bonded to a microscope glass slide (coated with (3-Aminopropyl)triethoxysilane (APTES)) under oxygen-plasma treatment.

### Surface Functionalization

The microfluidic channel is rinsed with 100% ethanol, and is incubated with (3-Mercaptopropyl)trimethoxysilane (MPTMS, 20% in ethanol) for 15 min in room temperature (RT). Thereafter, the microchannel is rinsed with 100% ethanol and incubated with N-γ-maleimidobutyryl-oxysuccinimide ester (GMBS, 0.28% in ethanol) for 15 min in RT. The microchannel is rinsed with 100% ethanol and phosphate buffered saline (PBS), and is incubated with laminin (25 μg/mL in PBS) for 1.5 h in RT. Finally, the microchannel is incubated with bovine serum albumin (BSA, 2% in PBS) at 4° C. overnight.

### Testing

The microfluidic channel is connected to a constant pressure pump. A 20% hematocrit RBC suspension is perfused through the microchannel at a constant inlet pressure of 35 mBar for 20 min, after which the microchannel is washed with PBS for 30 min. Microcapillary occlusion and RBC adhesion events are quantified within the area of interest, which is the area containing the last four micropillar arrays.

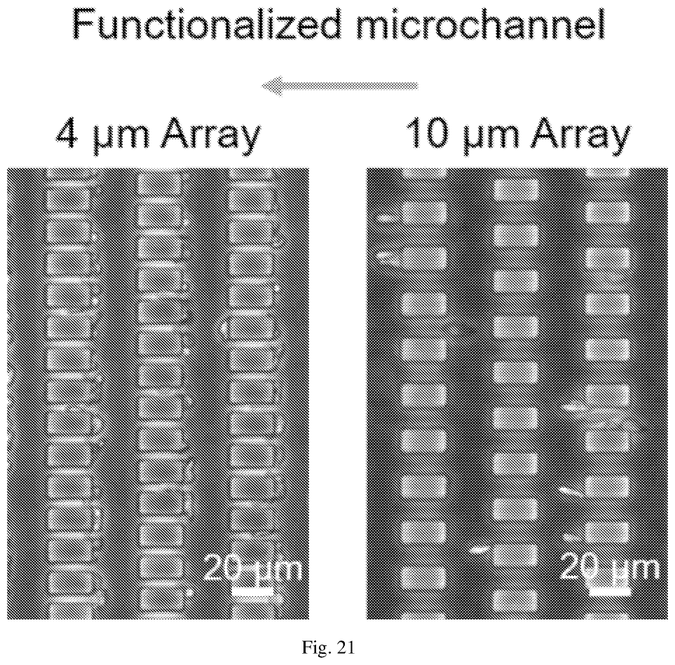

To test the concept of the microfluidic device, RBCs were isolated from a clinical blood sample from a subject with homozygous SCD and were tested using a functionalized microchannel with laminin and a non-functionalized microchannel. Results show that in the laminin-functionalized microchannel, apart from that pathological RBCs physically interacted with the microchannel geometries and induced microcapillary occlusion at the downstream, similar to what is observed in the non-functionalized channel (not shown), pathological RBCs adhered to the microchannel walls (on micropillars or bottom surfaces) (FIG. 21). These results demonstrated that the proposed microfluidic device can be utilized for concurrent assessment of RBC adhesion and microvascular occlusion.

### Example 3

A Microfluidic Approach for Rapid Separation of Plasma from Whole Blood

Blood tests are one of the most widespread types of medical laboratory diagnostics, which are normally performed on plasma and heavily dependent on laboratory infrastructures such as centrifugation. The existence of blood cells in plasma can significantly challenge assays on various plasma proteins and lead to inaccurate quantification. Therefore, the development of a simple, inexpensive, and rapid on-chip plasma separation approach is greatly demanded to facilitate point-of-care (POC) testing which can be conducted in a resource-constrained environment.

In response to the need, here we developed a simple microfluidic approach to separate plasma from whole blood for rapid diagnostic tests (RDTs). RDTs have offered great potentials to improve the diagnosis of malaria, and many RDTs have been focusing on the detection of Plasmodium falciparum histidine rich protein (HRP) II (pfHRP-2), which is a major target antigen. Plasma pfHRP-2 is significantly associated with the progression of the disease, however, inclusion of red blood cells (RBCs) in plasma may negatively affect the result of pfHRP-2-based RDTs when predicting disease severity of malaria, which is caused by the high level and the large variability of pfHRP-2 within RBCs. Hence, we identified two primary design criteria in this work:
1—Rapid separation of plasma from a small volume of finger-stick whole blood
2—Prevention of RBC lysis and consequent pfHRP-2 release to the plasma.

With these criteria in mind, we designed a method that: (i) accelerates the sedimentation of blood cells to facilitate the separation of cell-free plasma and reduce the burden of the on-chip filtration of red blood; and (ii) induce structural stiffening of RBCs that will help with microfluidic filtering in a manner no damage is imposed on RBCs.

### Experimental

The experiments are performed immediately after blood draw through a finger-stick. We examine the effect of additional fibrinogen on RBC sedimentation, which has been reported to have a marked correlation between the concentration of and the erythrocyte sedimentation rate.

### Results

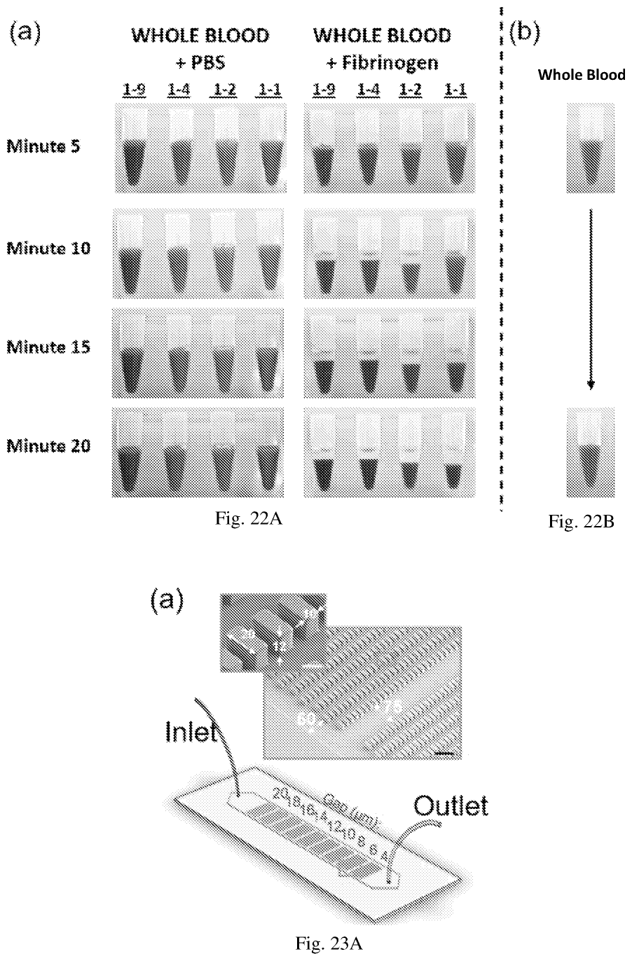

Whole venous blood was drawn with anticoagulated EDTA into a 1.5 ml microcentrifuge tube. Fibrinogen stock solution with the concentration of 40 mg/ml (in PBS) was mixed with whole blood at ratios of 1:9, 1:4, 1:2, and 1:1 (v/v) and the final volume of the mixtures was adjusted to 500 μl. Control groups were mixtures of whole blood and PBS at the same volume ratios. The sedimentation of RBCs was monitored over a period of 20 minutes. Sedimentation of RBCs in pre-processed whole blood was also observed, FIG. 22A. shows the level of RBC sedimentation of samples with different concentrations of fibrinogen as well as the control groups at the time points of 5 min, 10 min, 15 min and 20 min. FIG. 22B shows a negligible RBC sedimentation level when whole blood was used without the addition of another substance. Visually, fibrinogen significantly accelerated the sedimentation of RBCs compared to PBS. In addition, fibrinogen mediated RBC sedimentation became stable when it approached to 20 min, and no more noticeable difference was observed between blood samples with different concentrations of fibrinogen. Therefore, the 1:9 fibrinogen and whole blood mixture (4 mg/ml final fibrinogen concentration) was selected based on a cost-effective point of view, from which 150 μl plasma was collected for further processing.

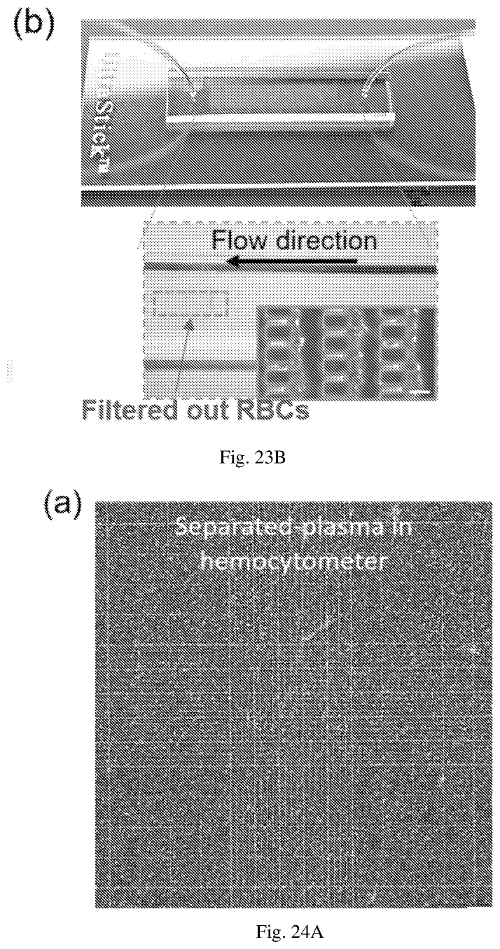

The experiments can be performed with a microfluidic device described herein, in which series of micropillar arrays forming various micro constrictions ranging from 20 μm to 4 μm were embedded for RBC deformability study (FIG. 23). Using this device, we recently found that a spectrin-specific cross-linker, diamide, is able to induce structural stiffening of RBCs without interrupting the cell membrane morphology. We hypothesized that following the structural stiffening of RBCs within the separated plasma through diamide exposure, the poorly deformable RBCs can be filtered by our microfluidic device.

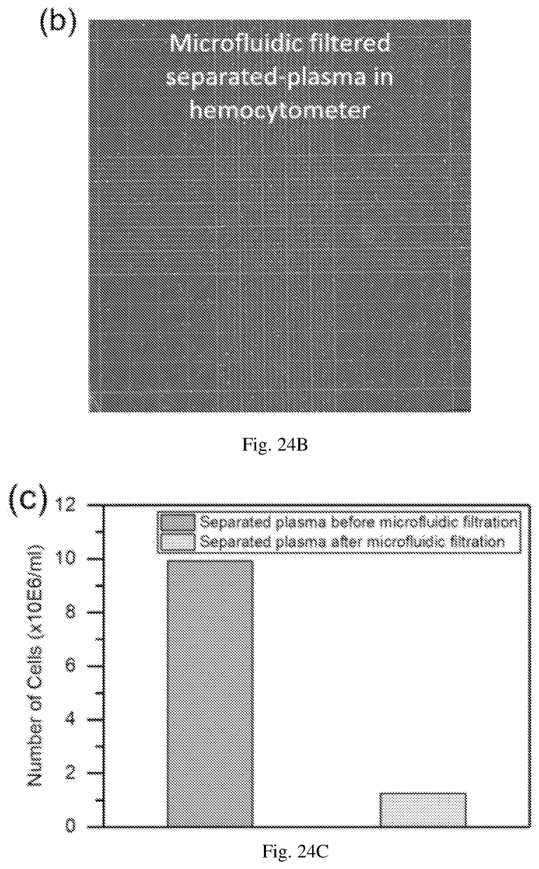

The microfluidic device was fabricated based on standard photolithography and soft lithography. A negative template on a 3-inch silicon wafer was first fabricated. Briefly, a layer of negative photoresist SU8-2010 was initially spin-coated on top of the wafer. After soft-baked at 95° C. for 4 min, the wafer was exposed to UV light with alignment to selectively cure the photoresist. Following the post-exposure bake, the wafer was developed in a photoresist solvent propylene glycol monomethyl ether acetate (PGMEA) and hard-baked at 110° C. overnight. A 2-hour surface passivation using trichloro (1H, 1H, 2H, 2H-perfluorooctyl) saline was performed under vacuum to facilitate the separation of the molded polymer from the master wafer. Next, PDMS pre-polymer was mixed with the curing agent at a ratio of 10:1 (v/v) and degassed in a desiccator to remove any air bubbles. The mixture was poured over the master wafer and cured at 80° C. overnight. Two 0.5 mm-diameter holes were punched as the inlet and the outlet after the PDMS block was peeled-off from the master wafer. Excessive saline was removed by sonicating with isopropanol for 5 min. Tubing was assembled after the PDMS block was bonded to a microscope glass slide through the surface modification under oxygen-plasma treatment. The inner surface of the microchannel was passivated by bovine serum albumin at 4° C. overnight. The separated plasma in specific aim 1 was incubated with 0.08% (w/v) diamide at 37° C. for 5 min and then perfused through the microchannel at a constant inlet pressure. The filtered plasma was collected at the outlet. 10 μl of plasma before and after the microfluidic filtration were examined using a hemacytometer (FIGS. 24A & B). Our result indicates a more than 7-fold decrease of cell number after filtration (FIG. 24C).

We found addition of fibrinogen into whole blood accelerates RBC sedimentation, which aids in collection of relatively large volume of plasma, and our microfluidic device effectively filters stiffened RBCs to a significant extent.

Based on the data generated herein, and the specific needs outlined, we will re-design our microfluidic device to narrow down the micro constrictions to 3 μm and eliminate the large openings on the two sides of the microchannel in order to increase the efficiency of RBC filtration for RDT based pfHRP-2 testing.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: A microfluidic device comprising:
at least one microchannel that extends through a portion of a housing, the at least one microchannel being configured to receive a fluid sample that flows along a length of the microchannel from a first end to a second end of the microchannel, the at least one microchannel including a plurality of micropillar arrays provided along the length of the microchannel, wherein each micropillar array defines a plurality microcapillaries having a width and cross sectional area and the width and/or cross sectional area of the microcapillaries defined by each micropillar array decreases in a direction of fluid flow through the microchannel.

2: The microfluidic device of claim 1, the microchannel including a substantially planar upper surface and a substantially planar lower surface, micropillars of the plurality of micropillar arrays extending from upper surface to the lower surface.

3: The microfluidic device of claim 1, wherein each of the micropillars of the plurality of micropillar arrays has a substantially rectangular cross section.

4: The microfluidic device of claim 1, wherein each of the microcapillaries has a substantially rectangular cross section.

5: The microfluidic device of claim 1, wherein the microchannel includes at least three micropillar arrays and the widths and cross sectional areas of the microcapillaries defined by each respective micropillar array being substantially uniform.

6: The microfluidic device of claim 1, wherein each micropillar array includes at least three rows of micropillars, the rows extending perpendicular to fluid flow and having a substantially similar shape.

7: The microfluidic device of claim 6, the distance between each micropillar in a row of a respective micropillar array is substantially the same.

8: The microfluidic device of claim 1, wherein successive micropillar arrays are separated from each other in the microchannel by gap region, the gap region being free of micropillars.

9: The microfluidic device of claim 1, wherein the microchannel includes a micropillar array at the first end that defines a plurality of microcapillaries that each have a width of about 18 μm to about 22 μm and/or a cross sectional area of about 200 μm² to about 250 μm² and each successive micropillar array in the direction of fluid flow through the microchannel defines a plurality of microcapillaries that each have a width and/or cross sectional area about 5% to about 50% less than a plurality of microcapillaries defined by a preceding micropillar array.

10: The microfluidic device of claim 9, wherein the microchannel includes a micropillar array at the second end that defines a plurality of microcapillaries that each have a width of about 3 μm to about 6 μm and/or a cross sectional area of about 40 μm² to about 50 μm² and each preceding micropillar array in opposite the direction of fluid flow through the microchannel defines a plurality of microcapillaries that each have a width and/or a cross sectional area about 5% to about 50% greater than a plurality of microcapillaries defined by a preceding micropillar array.

11: The microfluidic device of claim 1, including at least eight micropillar arrays, the configured along the length of the microchannel, a micropillar array at the first end defining a plurality of microcapillaries that each have a width of about 18 μm to about 22 μm and a cross sectional area of about 200 μm² to about 250 μm² and a micropillar array at the second end that defines a plurality of microcapillaries that each have a width of about 3 μm to about 5 μm and a cross sectional area of about 40 μm² to about 50 μm².

12: The microfluidic device of claim 1, wherein the width and/or cross sectional area of the plurality of microcapillaries defined by at least one of the plurality of micropillar arrays permits passage of healthy cells in a fluid sample perfused through the microchannel but occludes cells with impaired deformability.

13: The microfluidic device of claim 1, wherein the fluid sample includes blood cells.

14: The microfluid device of claim 13, wherein the cells are red blood cells.

15: The microfluidic device of claim 1, wherein the width and/or cross sectional area of the plurality of microcapillaries at the second of the microchannel occludes cells in a fluid sample perfused through the microchannel.

16: The microfluidic device of claim 1, wherein each of the micropillar arrays is arranged in an inner portion of the microchannel that extends the length of the microchannel, the microchannel including two parallel outer passages on opposite sides of the inner portion that extend the length of the microchannel, outer passages being in fluid communication with the plurality of microcapillaries defined by the plurality micropillar arrays.

17: The microfluidic device of claim 16, wherein the outer passages have cross sectional areas that permit cells in a fluid sample to flow through the microchannel without being occluded and/or obstructed.

18: The microfluidic device of claim 1, the microchannel including a substantially planar transparent wall that defines the upper surface or lower surface of the microchannel.

19: The microfluidic device of claim 18, wherein the substantially planar transparent wall permits observation into the microfluidic channel by microscopy.

20: The microfluidic device of claim 1 further comprising a micro-gas exchanger for controlling the oxygen content of the blood prior to and/or after delivering the blood to the at least one microchannel.

21: The microfluidic device of claim 20, the micro-gas exchanger providing hypoxic blood to the at least one microchannel.

22: The microfluidic device of claim 1, further comprising at least one capturing agent that is immobilized on a surface of the at least one microchannel, the capturing agent adhering a cell of interest to the at least one surface of the at least one microchannel when a fluid sample containing cells is passed through the at least one microchannel.

23: The microfluidic device of claim 22, the at least one capturing agent comprising at least one of laminin, fibronectin, E-Selectin, P-Selectin, L-selectin, intracellular adhesion molecule 1 (ICAM-1), or vascular cellular adhesion molecule 1 (VCAM-1).

24: The microfluidic device of claim 22, the capturing agent being covalently immobilized to at least one surface of the at least one microchannel with a cross-linker.

25: The microfluidic device of claim 24, the cross-linker being GMBS.

26-51. (canceled)
